(19) United States
(12) Patent Application Publication
(10) Pub. No.: US 2024/0226100 A1
(43) Pub. Date: Jul. 11, 2024

(54) CONTROL OF PROTEIN EXPRESSION WITH TMP-PROTAC COMPOUNDS

(71) Applicant: THE TRUSTEES OF THE UNIVERSITY OF PENNSYLVANIA, Philadelphia, PA (US)

(72) Inventors: Mark A. SELLMYER, Philadelphia, PA (US); Iris Kyungmin LEE, Seoul (KR); Andrew RUFF, New York, NY (US); Nitika SHARMA, Philadelphia, PA (US); Jean M. ETERSQUE, Philadelphia, PA (US); Justin NORTHRUP, Langhorne, PA (US)

(21) Appl. No.: 18/554,521

(22) PCT Filed: Apr. 11, 2022
(86) PCT No.: PCT/US2022/071660

§ 371 Date: Oct. 9, 2023

Related U.S. Application Data
(60) Provisional application No. 63/173,087, filed on Apr. 9, 2021.

(51) Int. Cl. A61K 31/506; A61K 49/04; C07D 401/14; C12N 9/06

(52) U.S. Cl. CPC A61K 31/506; A61K 49/0442; C07D 401/14; C12N 9/003; C12Y 105/01003

(57) ABSTRACT

Described are compounds of formula (I), or pharmaceutically acceptable salts thereof: (I) that are useful for control of protein expression with eDHFR tags and methods of controlling protein expression with such compounds, as well as kits comprising the compounds.

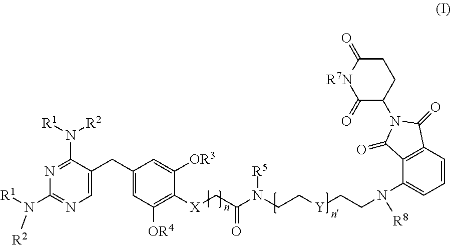

### CROSS-REFERENCE TO RELATED APPLICATIONS

This application claims the benefit of U.S. Provisional Application No. 63/173,087, filed Apr. 9, 2021, the disclosure of which is incorporated herein by reference in its entirety.

### TECHNICAL FIELD

The disclosure is directed to TMP-PROTAC compounds useful for control of protein expression with eDHFR tags and methods of controlling protein expression with such compounds, as well as kits comprising the TMP-PROTAC compounds.

### BACKGROUND OF THE INVENTION

The ability to tunably and reversibly regulate protein function and expression is a critical goal for basic inquiry into the biochemical function(s) of proteins in cells as well as for the next generation of translational therapeutics. Many genetic approaches to engineering this control such as knockouts, transcriptional activators or repressors, and RNAi are available but have some unique and shared limitations, for example ribonucleic acid delivery to target tissues in animals. Small molecule approaches have some of the best translational properties for in vivo absorption and delivery to all tissues. These approaches include direct inhibition of a protein through drug discovery/medicinal chemistry efforts and newer methods that impact protein function via protein expression regulation. These new methods use proteolysis targeted chimeric small molecules (PROTACs) and a combined chemical and genetic approach using generalizable protein tags called destabilizing domains (DDs). The development of such small protein tags that can be appended to any protein of interest (POI) opens the door for currently undruggable, chimeric, or other protein classes to be tunably regulated. DDs have been developed which include domains based on FK-506 binding protein (FKBP12 F36V), the bacterial enzyme E. coli dihydrofolate reductase (eDHFR), and the estrogen receptor (ER). Dihydrofolate reductase from E. coli (eDHFR) is a small protein tag that is genetically ligated to a protein of interest (POI). The expressed protein complex is regulatable with a bifunctional drug. In the absence of drug, the protein complex is active, but in the presence of drug, the protein complex is degraded via proteosome-mediated degradation and is no longer active. PROTACs controlling small protein tags also have been developed for the bacterial Halo-Tag and FKBP12 F36V, and the IKZF3 ZF2 domain has been used as a degron to engender post-translational regulation of membrane proteins based on the presence of an immunomodulatory imide drug (IMiD), lenalidomide.

The fundamental difference between these standard approaches is that the DDs are best-suited as a drug-ON approach, where the binding of the small molecule favorably impacts the stability of the protein, thus increasing the half-life of the protein in the cell. PROTACs are a drug-OFF system whereby the chimeric small molecule binding forms a ternary complex between the small protein tag and an E3 ligase capable of driving ubiquitination of the fusion protein, which targets it for degradation. Thus, PROTAC binding decreases the cellular half-life of the protein and reduces protein levels. For translational therapeutic protein control, the drug-OFF techniques may be favorable as they can be employed only in selected situations such prevention or abrogation of toxicity related to the protein expression derived from the gene or cell therapy, rather than drug-ON systems that require dosage at regular intervals to maintain therapeutic efficacy.

There exists a need in the art for compounds that can effectively and efficiently target and regulate a protein.

### SUMMARY OF THE INVENTION

The disclosure provides compounds that can effectively and efficiently target and regulate a protein, and methods of preparing the compounds and methods of using the compounds.

In some embodiments, provided is a TMP-PROTAC compound that is a compound of formula (I):

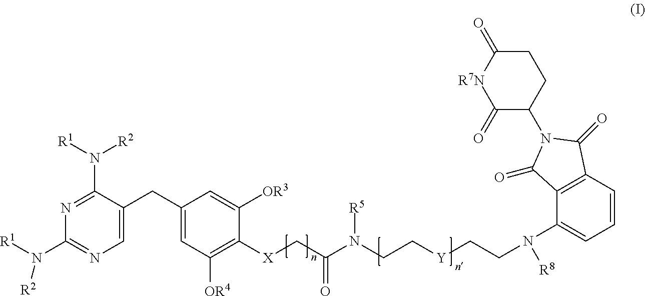

or a pharmaceutically acceptable salt thereof, wherein:
X is —O—, —S—, —CR¹R²— or —NR¹—;
Y is —O—, —S—, —CR¹R²— or —NR¹—;
each of R¹, R², R³, R⁴, R⁵, R⁶ and R⁷ is independently selected from hydrogen or C₁-C₆ alkyl;
n is 1, 2, 3, 4, 5, or 6; and
n′ is 1, 2, 3, 4, 5 or 6.

In some embodiments, X and Y are both —O— and each of R¹, R², R³, R⁴, R⁵, R⁶ and R⁷ is hydrogen. In some embodiments, the compound of formula I is compound 7a, 7b, 7c or 7e.

In some embodiments, provided is a method of regulating protein expression comprising contacting dihydrofolate reductase enzyme (DHFR) with a compound of formula (I) or pharmaceutically acceptable salt thereof. In some embodiments, the DHFR is Escherichia coli dihydrofolate reductase enzyme (eDHFR).

In some embodiments, provided is a method of degrading a protein of interest comprising contacting the protein of interest with a compound of formula (I) or pharmaceutically acceptable salt thereof.

In some embodiments, provided is a method of making an engineered cell comprising fusing a protein of interest to dihydrofolate reductase enzyme (DHFR) with a compound of formula (I) or pharmaceutically acceptable salt thereof.

In some embodiments, the protein of interest is a kinase, a cytokine, an immunotherapy protein, a chimeric protein, a structural protein, a transcription factor, a hormone, a growth factor, an immunoglobulin (e.g., antibody), an immunoglobulin-like domain-containing molecule (e.g., an ankyrin or a fibronectin domain-containing molecules), and an Fc-fusion protein.

In some embodiments, the protein of interest is a chimeric antigen receptor (CAR), yellow fluorescent protein (YFP) or luciferase. In some embodiments, provided is a degraded protein made by the methods described herein.

In some embodiments, provided is a kit comprising a dihydrofolate reductase enzyme (DHFR) construct and a compound of formula (I) or pharmaceutically acceptable salt thereof. In some embodiments, the DHFR is Escherichia coli dihydrofolate reductase enzyme (eDHFR).

In some embodiments, provided is a method of in vivo imaging a mammalian cell comprising the steps of:
(a) administering

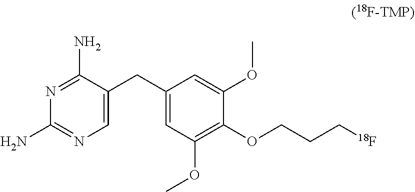 [SMILES: COc1cc(Cc2cnc(N)nc2N)cc(OC)c1OCCC[18F]]

to a subject; and
(b) imaging in vivo distribution of the ¹⁸F-TMP by positron emission tomography (PET) scanning.

### BRIEF DESCRIPTION OF THE DRAWINGS

FIG. 1 depicts time and dose-dependent degradation of YFP in Jurkat-DYL using compounds 7a, 7b, 7c and 7e.

FIG. 2 depicts degradation of eDHFR-POI using compounds 7a, 7b, 7c and 7e.

FIG. 3 depicts degradation of luciferase using eDHFR/compound 7c.

FIG. 4 depicts time and dose-dependent degradation of YFP and Luciferase in human embryonic kidney HEK293T/17 cells using compound 7c.

FIG. 5 depicts degradation of CAR molecules from the surface of CAR T cells using compound 7c.

FIG. 6 depicts “cell signaling changes” related to degradation of CAR molecules from the surface of Jurkat cells using compound 7c.

FIG. 7 depicts dose response and time course of 7c in Jurkat^{eDHFR-YFP+} cells.

FIG. 8 depicts reversal kinetics of YFP degradation in Jurkat^{eDHFR-YFP} cells.

FIG. 9 depicts degradation of eDHFR-YFP in HEK293T (HEK293T^{eDHFR-YFP+}) cells analyzed by Western blot with anti-YFP antibody.

FIG. 10 depicts dose response in HEK293T^{eDHFR-YFP+} cells with compound 7c at 24 h.

FIG. 11 depicts time course in HEK293T^{eDHFR-YFP+} cells with compound 7c at 6, 12 and 24 h.

FIG. 12 depicts Western blot analysis of eDHFR-YFP recovery in HEK293T^{eDHFR-YFP+} cells incubated with 100 nM 7c, washed twice with PBS, then replenished with new media.

FIG. 13 depicts western blot characterization of proteasome degradation mechanism in HEK293T^{eDHFR-YFP+} cells.

FIG. 14 depicts HEK293T^{eDHFR-YFP+} cells were incubated with either 500 nM MLN4924 or 25 μM 3-Methyladenine for 1 h, followed by the addition of, 100 nM of 7c, 25 μM TMP or 2.5 μM Pomalidomide, where cells were incubated for an additional 12 h.

FIG. 15 shows characterization of 7f by Western blot analysis with anti-YFP antibody.

FIG. 16 depicts dose response in HEK293T^{+eDHFR-Lck} cells with compound 7c at 24 h.

FIG. 17 depicts dose response in HEK293T^{+eDHFR-RUX1} cells with compound 7c at 24 h.

FIG. 18 depicts dose response in HEK293T^{+CD122-eDHR} cells with compound 7c at 24 h.

FIG. 19 depicts OVCAR8 cells expressing eDHFR-luc (OVCAR8eDHFR-luc+) were incubated with compound 7c for 4-48 h.

FIG. 20 depicts TMP-POM 7c PROTAC effectively downregulates CAR in a dose-dependent and reversible manner.

FIG. 21 depicts downregulation of CAR with TMP-POM PROTAC inhibits CAR T cell signaling and its cytotoxic function against target cells in vitro.

FIG. 22 depicts TMP-POM 7c can modulate the cytotoxic activity of FAP-eDHFR DF CAR T cells in a dose-dependent manner with TMP-POM 7c.

FIG. 23 depicts In vitro characterization of FAP-eDHFR DF CAR constructs.

FIG. 24 depicts dose response assay with N-Methyl 7c (7f).

FIG. 25 depicts comparison of cytotoxic function of different eDHFR-expressing FAP CAR T cells.

FIG. 26 depicts downregulation of CAR by TMP-POM 7C PROTAC is a proteosome-mediated degradation process.

FIG. 27 depicts evaluation of the “imageability” of FAP-eDHFR Direct Fusion (DF) CAR T cells.

### DETAILED DESCRIPTION OF ILLUSTRATIVE EMBODIMENTS

The disclosure may be more fully appreciated by reference to the following description, including the following definitions and examples. Certain features of the disclosed processes are described herein in the context of separate aspects, may also be provided in combination in a single aspect. Alternatively, various features of the disclosed processes that are, for brevity, described in the context of a single aspect, may also be provided separately or in any sub-combination.

The ability to tunably and reversibly regulate protein function and expression is a critical goal for basic inquiry into the biochemical function.

eDHFR can be used to image and regulate CAR T cells depending on how its ligand TMP is functionalized; radiolabeled TMP allows for imaging and tracking of CAR T cells with nuclear imaging, while functionalized TMP-Pomalidomide (TMP-POM) PROTAC allows for targeted degradation of CAR from the surface. TMP was derivatized at the methoxy group para to the pyrimidine ring and was attached to pomalidomide via a PEG linker. eDHFR protein was directly fused to the C-terminus of CD3zeta domain of FAP CAR construct to allow for regulation with TMP-POM PROTAC.

Here we report the development of PROTACs for the bacterial protein eDHFR using chimeric small molecules that are comprised of trimethoprim, varied chemical linkers, and pomalidomide. Pomalidomide is a small molecule inhibitor that targets the cereblon E3 ligase, with approximate 3 micromolar affinity and has been used successfully in numerous PROTACs. Trimethoprim has a low-nanomolar affinity for eDHFR. We show in immortalized cell lines tunable and dose-dependent regulation of optical proteins such as fluorescent proteins and luciferase. Furthermore, we show regulation of a chimeric antigen receptor (CAR) using TMP-PROTACs in Jurkats and primary human T-cells. Downregulation of CAR protein with the optimized TMP-PROTAC led to abrogation of primary human CAR T cell cytokine signaling and killing of target cells in vitro. This work dovetails with on-going efforts in our lab to image the location of genetically engineered cells such as CAR T cells using radiotracers based on trimethoprim, and provides a new drug-OFF, PROTAC mediated protein tag. We envision this work will be broadly applicable to basic science investigators looking for drug-OFF protein regulation systems and translational therapeutic approaches where chemical control of a therapeutic protein is critical.

The compounds of formula (I) described herein are TMP-PROTACs based on trimethoprim (TMP) and pomalidomide, a known CRBN E3 ligase inhibitor, with variation in linker length.

In some embodiments, the disclosure is directed to a compound of Formula (I):

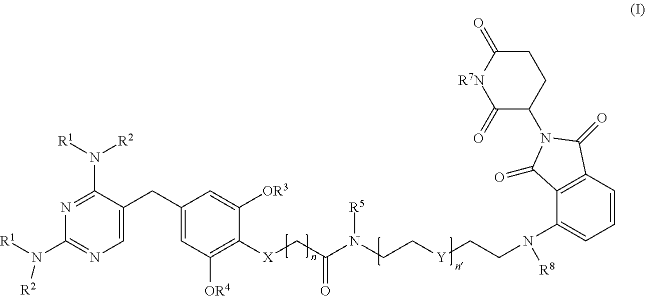

or a pharmaceutically acceptable salt thereof, wherein:
X is —O—, —S—, —CR¹R²— or —NR¹—;
Y is —O—, —S—, —CR¹R²— or —NR¹—;
each of R¹, R², R³, R⁴, R⁵, R⁶ and R⁷ is independently selected from hydrogen or C₁-C₆ alkyl;
n is 1, 2, 3, 4, 5, or 6; and
n′ is 1, 2, 3, 4, 5 or 6.

In some embodiments, X of formula (I) is —O—, —S—, —CR¹R²— or —NR¹—. In some embodiments, X is —O—. In some embodiments, X is —S—. In some embodiments, X is —CR¹R²—. In some embodiments, X is —NR¹—.

In some embodiments, Y of formula (I) is —O—, —S—, —CR¹R²— or —NR¹—. In some embodiments, Y is —O—. In some embodiments, Y is —S—. In some embodiments, Y is —CR¹R²—. In some embodiments, Y is —NR¹—.

In some embodiments, both X and Y of formula (I) are —O—.

In some embodiments, R¹, R², R³, R⁴, R⁵, R⁶ and R⁷ of formula (I) are independently selected from hydrogen or C₁-C₆ alkyl.

In some embodiments, at least one of R¹, R², R³, R⁴, R⁵, R⁶ and R⁷ of formula (I) is hydrogen. In some embodiments, each of R¹, R², R³, R⁴, R⁵, R⁶ and R⁷ of formula (I) is hydrogen.

In some embodiments, at least one of R¹, R², R³, R⁴, R⁵, R⁶ and R⁷ of formula (I) is C₁-C₆ alkyl. In some embodiments, each of R¹, R², R³, R⁴, R⁵, R⁶ and R⁷ of formula (I) is C₁-C₆ alkyl. In some embodiments, the C₁-C₆ alkyl is selected from methyl, ethyl or isopropyl. In some embodiments, the C₁-C₆ alkyl is methyl. In some embodiments, the C₁-C₆ alkyl is ethyl. In some embodiments, the C₁-C₆ alkyl is isopropyl.

In some embodiments, n of formula (I) is 1, 2, 3, 4, 5, or 6. In some embodiments, n is 1. In some embodiments, n is 2. In some embodiments, n is 3. In some embodiments, n is 4. In some embodiments, n is 5. In some embodiments, n is 6.

In some embodiments, n′ of formula (I) is 1, 2, 3, 4, 5, or 6. In some embodiments, n′ is 1. In some embodiments, n′ is 2. In some embodiments, n′ is 3. In some embodiments, n′ is 4. In some embodiments, n′ is 5. In some embodiments, n′ is 6.

In some embodiments, n of formula (I) is 1 and n′ of formula (I) is 2. In some embodiments, n of formula (I) is 3 and n′ of formula (I) is 1. In some embodiments, n of formula (I) is 3 and n′ of formula (I) is 2. In some embodiments, n of formula (I) is 3 and n′ of formula (I) is 6.

In some embodiments, the compound of formula (I) is:

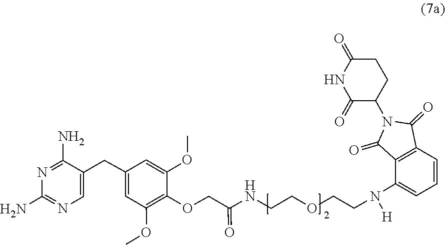

or a pharmaceutically acceptable salt thereof.

In some embodiments, the compound of formula (I) is:

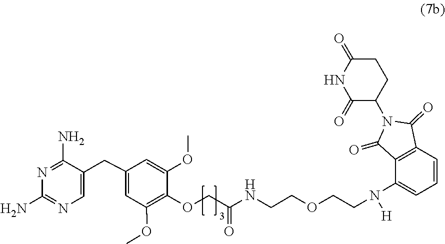

or a pharmaceutically acceptable salt thereof.

In some embodiments, the compound of formula (I) is:

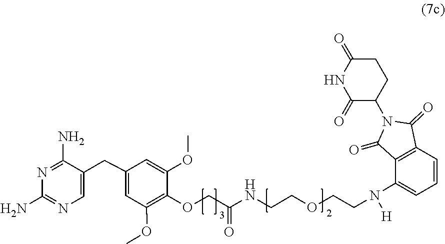

or a pharmaceutically acceptable salt thereof.

In some embodiments, the compound of formula (I) is:

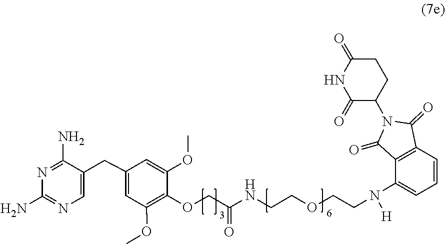

or a pharmaceutically acceptable salt thereof.

In some embodiments, the compound of formula (I) is:

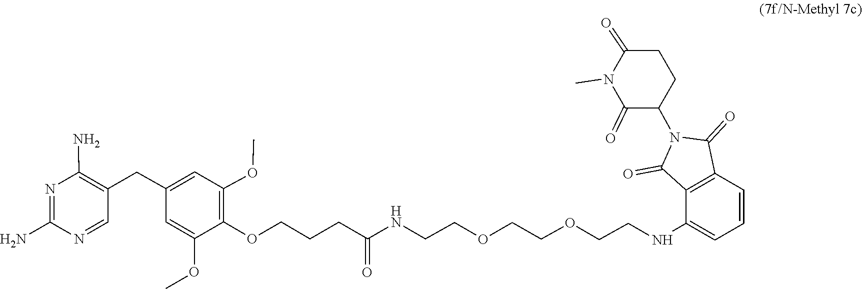 [SMILES: COc1cc(Cc2cnc(N)nc2N)cc(OC)c1OCCCC(=O)NCCOCCOCCNc1cccc2c1C(=O)N(C1CCC(=O)N(C)C1=O)C2=O]

or a pharmaceutically acceptable salt thereof.

In some embodiments, the disclosure is directed to methods of using compounds of Formula (I).

In some embodiments, the disclosure is directed to methods of regulating protein expression of a mammalian cell comprising contacting dihydrofolate reductase enzyme (DHFR) with a compound of formula (I) or pharmaceutically acceptable salt thereof. In some embodiments, the compound of formula (I) or pharmaceutically acceptable salt thereof binds to the DIFR. In some embodiments, the DIFR is Escherichia coli dihydrofolate reductase enzyme (eDHFR).

In some embodiments, the methods of regulating protein expression further comprise in vivo imaging of the mammalian cell comprising the steps of: (a) administering

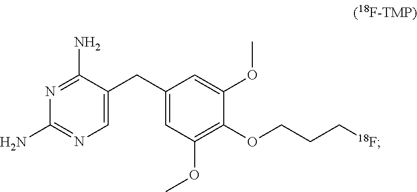

and (b) imaging in vivo distribution of the ¹⁸F-TMP by positron emission tomography (PET) scanning.

In some embodiments, the disclosure is directed to methods of degrading a protein of interest comprising contacting the protein of interest with a compound of formula (I) or pharmaceutically acceptable salt thereof.

In some embodiments, the protein of interest is a kinase, a cytokine, an immunotherapy protein, a chimeric protein, a structural protein, a transcription factor, a hormone, a growth factor, an immunoglobulin (e.g., antibody), an immunoglobulin-like domain-containing molecule (e.g., an ankyrin or a fibronectin domain-containing molecule), and an Fc-fusion protein.

In some embodiments, the protein of interest is a kinase. In some embodiments, the protein of interest is a cytokine. In some embodiments, the protein of interest is an immunotherapy protein. In some embodiments, the protein of interest is a chimeric protein. In some embodiments, the protein of interest is a structural protein. In some embodiments, the protein of interest is a transcription factor. In some embodiments, the protein of interest is a hormone. In some embodiments, the protein of interest is a growth factor. In some embodiments, the protein of interest is an immunoglobulin. In some embodiments, the protein of interest is an antibody. In some embodiments, the protein of interest is an immunoglobulin-like domain-containing molecule. In some embodiments, the protein of interest is an ankyrin. In some embodiments, the protein of interest is a fibronectin domain-containing molecule. In some embodiments, the protein of interest is an Fc-fusion protein.

In some embodiments, the protein of interest is a chimeric antigen receptor (CAR), yellow fluorescent protein (YFP) or luciferase. In some embodiments, the protein of interest is a chimeric antigen receptor (CAR). In some embodiments, the protein of interest is a yellow fluorescent protein (YFP). In some embodiments, the protein of interest is a luciferase.

In some embodiments, provided is a degraded protein made by the methods described herein.

In some embodiments, the protein of interest is located in an engineered cell. In some embodiments, the engineered cell comprises dihydrofolate reductase enzyme (DHFR) fused to the protein of interest. In some embodiments, the DHFR is Escherichia coli dihydrofolate reductase enzyme (eDHFR). In some embodiments, a degraded protein is made by the methods of protein degradation described herein. In some embodiments, the DIFR is genetically fused to the protein of interest.

In some embodiments, the disclosure is directed to methods of making an engineered cell comprising fusing a protein of interest to dihydrofolate reductase enzyme (DHFR) with a compound of formula (I), or pharmaceutically acceptable salt thereof.

In some embodiments, the protein of interest is a kinase, a cytokine, an immunotherapy protein, a chimeric protein, a structural protein, a transcription factor, a hormone, a growth factor, an immunoglobulin (e.g., antibody), an immunoglobulin-like domain-containing molecule (e.g., an ankyrin or a fibronectin domain-containing molecule), and an Fc-fusion protein.

In some embodiments, the protein of interest is a kinase. In some embodiments, the protein of interest is a cytokine. In some embodiments, the protein of interest is an immunotherapy protein. In some embodiments, the protein of interest is a chimeric protein. In some embodiments, the protein of interest is a structural protein. In some embodiments, the protein of interest is a transcription factor. In some embodiments, the protein of interest is a hormone. In some embodiments, the protein of interest is a growth factor. In some embodiments, the protein of interest is an immunoglobulin. In some embodiments, the protein of interest is an antibody. In some embodiments, the protein of interest is an immunoglobulin-like domain-containing molecule. In some embodiments, the protein of interest is an ankyrin. In some embodiments, the protein of interest is a fibronectin domain-containing molecule. In some embodiments, the protein of interest is an Fc-fusion protein.

In some embodiments, the protein of interest is a chimeric antigen receptor (CAR), yellow fluorescent protein (YFP) or luciferase. In some embodiments, the protein of interest is a chimeric antigen receptor (CAR). In some embodiments, the protein of interest is a yellow fluorescent protein (YFP). In some embodiments, the protein of interest is a luciferase.

In some embodiments, the DIFR is Escherichia coli dihydrofolate reductase enzyme (eDHFR). In some embodiments, an engineered cell is made by the methods of making an engineered cell described herein.

In some embodiments, the disclosure is directed to a kit comprising a dihydrofolate reductase enzyme (DHFR) construct and a compound of formula (I) or pharmaceutically acceptable salt thereof. In some embodiments, the DHFR is Escherichia coli dihydrofolate reductase enzyme (eDHFR).

In some embodiments, the disclosure is directed to methods of in vivo imaging of a mammalian cell comprising the steps of:
(a) administering

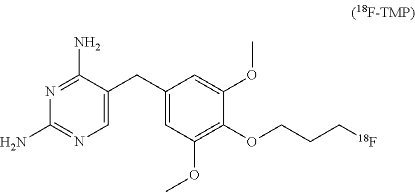 [SMILES: COc1cc(Cc2cnc(N)nc2N)cc(OC)c1OCCC[18F]]

to a subject; and
(b) imaging in vivo distribution of the ¹⁸F-TMP by positron emission tomography (PET) scanning.

In some embodiments, the cell is an engineered cell. In some embodiments, the engineered cell comprises dihydrofolate reductase enzyme (DHFR) fused to a protein of interest. In some embodiments, the DIFR is Escherichia coli dihydrofolate reductase enzyme (eDHFR). In some embodiments, the cell comprises a degraded protein.

In some embodiments, the protein of interest is a kinase, a cytokine, an immunotherapy protein, a chimeric protein, a structural protein, a transcription factor, a hormone, a growth factor, an immunoglobulin (e.g., antibody), an immunoglobulin-like domain-containing molecule (e.g., an ankyrin or a fibronectin domain-containing molecule), and an Fc-fusion protein.

In some embodiments, the protein of interest is a kinase. In some embodiments, the protein of interest is a cytokine. In some embodiments, the protein of interest is an immunotherapy protein. In some embodiments, the protein of interest is a chimeric protein. In some embodiments, the protein of interest is a structural protein. In some embodiments, the protein of interest is a transcription factor. In some embodiments, the protein of interest is a hormone. In some embodiments, the protein of interest is a growth factor. In some embodiments, the protein of interest is an immunoglobulin. In some embodiments, the protein of interest is an antibody. In some embodiments, the protein of interest is an immunoglobulin-like domain-containing molecule. In some embodiments, the protein of interest is an ankyrin. In some embodiments, the protein of interest is a fibronectin domain-containing molecule. In some embodiments, the protein of interest is an Fc-fusion protein.

In some embodiments, the protein of interest is a chimeric antigen receptor (CAR), yellow fluorescent protein (YFP) or luciferase. In some embodiments, the protein of interest is a chimeric antigen receptor (CAR). In some embodiments, the protein of interest is a yellow fluorescent protein (YFP). In some embodiments, the protein of interest is a luciferase.

In some embodiments, eDHFR, the protein target of the compounds of formula (I) are delivered with a vector selected from viral vectors (such as AAVs or oncolytic virus), Lenti viral vectors, retroviral vectors, naked DNA, mRNA, and engineered cells. In some embodiments, the compounds of formula (I) are delivered with a viral vector. In some embodiments, the viral vector is an adeno-associated virus (AAV). In some embodiments, the viral vector is an oncolytic virus. In some embodiments, the compounds of formula (I) are delivered with a Lenti viral vector. In some embodiments, the compounds of formula (I) are delivered with a retroviral vector. In some embodiments, the compounds of formula (I) are delivered with naked DNA. In some embodiments, the compounds of formula (I) are delivered with mRNA. In some embodiments, the compounds of formula (I) are delivered with engineered cells.

In some embodiments, the compounds of formula (I) are delivered to a cell selected from embryonic cells, endodermal cells, mesodermal cells, and ectodermal origin cells. In some embodiments, the compounds of formula (I) are delivered to an embryonic cell. In some embodiments, the compounds of formula (I) are delivered to an endodermal cell. In some embodiments, the compounds of formula (I) are delivered to a mesodermal cell. In some embodiments, the compounds of formula (I) are delivered to an ectodermal origin cell.

In some embodiments, the compounds of formula (I) are delivered to a cell selected from immune cells, stem cells, iPS cells, allogenic cells, autologous cells, mesenchymal cells and neurons. In some embodiments, the compounds of formula (I) are delivered to an immune cell. In some embodiments, the compounds of formula (I) are delivered to a stem cell. In some embodiments, the compounds of formula (I) are delivered to an iPS cell. In some embodiments, the compounds of formula (I) are delivered to an allogenic cell. In some embodiments, the compounds of formula (I) are delivered to an autologous cell. In some embodiments, the compounds of formula (I) are delivered to a mesenchymal cell. In some embodiments, the compounds of formula (I) are delivered to a neuron.

The following Examples are provided to illustrate aspects of the invention and are not intended to be limiting.

### EXAMPLES

### Materials and Methods

### Cell Culture

HEK293T cells (ATCC) were cultured in complete media: DMEM with 10% fetal bovine serum (Invitrogen), 2 mM glutamine, 100 U/mL penicillin and 100 mg/mL streptomycin (all from Gibco). Jurkat (ATCC) and OVCAR8 (ATCC) cells were cultured in complete media: RPMI with 10% fetal bovine serum (Invitrogen), 2 mM glutamine, 100 U/mL penicillin and 100 mg/mL streptomycin (all from Gibco). Cells were maintained in a humidified incubator at 37° C.

### Cloning and Generation of Stable Cell Lines

Stable cell lines expressing eDHFR-YFP-T2A-Luciferase (eDHFR-YFP) or eDHFR-Luciferase-T2A-mCherry (eDHFR-Luc) were generated by lentiviral transduction. eDHFR-YFP-T2A-Luc and eDHFR-Luc-T2A-mCherry genes were cloned into a pTRPE lentiviral vector backbone (gift of the Albelda and Pure lab at Penn), and lentivirus was packaged using HEK293T/17 (ATCC) and 2nd generation packaging plasmids (psPAX and pMD2). Target cells were transduced with lentivirus overnight in presence of 8ug/mL of polybrene, washed and incubated with fresh media for 1-2 days, passaged, and were sorted on either YFP (for eDHFR-YFP) or mCherry (for eDHFR-Luc) through fluorescence-activated cell sorting (BD).

### Mammalian Cell Dose Response Assay

HEK293T eDHFR-YFP (HEK293T^{eDHFR-YFP+}) and OVCAR8 eDHFR-luc (OVCAR8^{eDHFR-luc+}) cells are prepared in clear (Falcon) 6-well plates (5×10⁵ cells/well) and cultured in complete media. Compound 7c is solubilized in 100% DMSO to 10 mM. 10 mM 7c is serially diluted in sterile water accordingly and each dose administered to cells in fresh media at equal volume, such that the final concentration of DMSO in cell media is <1%. Once incubation with drug is completed, media is removed by vacuum, cells are washed with phosphate-buffered saline (PBS), trypsonized at 37° C., quenched with media, and centrifuged (Thermo Scientific Sorvall Legend X1R) at 1000 RPM for 5 minutes. Media and cell debris are removed by vacuum.

Likewise, Jurkat eDHFR-YFP (Jurkat^{eDHFR-YFP+}) cells are seeded in clear (Falcon) 12-well plates (3×10⁵ cells/well) in complete media. Compound 7c is added to each well at varying concentrations at −24, −8, and −4 h, and all samples were analyzed together on a flow cytometer (BD) at 0 h (t=0) to assess the degree of YFP expression.

### Preparation of Cell Lysate for Western Blot

Once cells are isolated by centrifugation (Thermo Scientific Sorvall Legend X1R), they are solubilized in radioimmunoprecipitation assay (RIPA) lysis buffer with protease inhibitor. Cell lysate is incubated on ice for 30 minutes, sonicated, then centrifuged (Thermo Scientific Sorvall Legend Micro 21R) at 14,800 RPM for 10 minutes. Supernatant is removed and transferred to a new Eppendorf tube.

### BCA Assay

Using Thermo Scientific bicinchoninic acid (BCA) assay kit, total cell protein is quantified and compared to bovine serum albumin (BSA) standards ranging from 10 to 0.625 mg/mL. Cell lysate is aliquoted into a 96-well clear plate (Falcon) and mixed with reagent and shaken at 37° C. for 30 minutes. Samples are analyzed by plate reader (ThermoFisher Varioskan Plusplate), where absorbance is measured at 480 nm. A calibration curve is developed, and samples are prepared to equal mass (mg) of total protein for gel electrophoresis.

### SDS-PAGE Gel

Cell lysate is prepared by mixing with 4 uL of loading dye and PBS to give equal total protein and equal total volume across all samples. Each sample is loaded into a NuPage gel (4-12% Bis-tris) and developed in NuPage MES Running Buffer. Once complete, the gel is removed and prepared for protein transfer to membrane.

### Protein Transfer

The SDS-page gel is prepared for protein transfer onto a polyvinylidene difluoride (PVDF) membrane (Biorad) that is activated by methanol, then layered into a transfer cassette. The cassette is loaded and developed in NuPage transfer buffer composed of 20% methanol at 4° C. for 1.5 h. Transfer is confirmed by Ponceau dye, which is washed and removed prior to antibody incubation.

### Antibody Incubation

PVDF membranes are blocked in 5% Milk/TBS for 1 h at room temperature, then rinsed gently with Tris-buffer saline (TBS)+1% Tween (TBST). Next, the membrane is incubated in primary antibody composed of 1:1000 antibody:5% Milk/TBS at 4° C. overnight. The membrane is rinsed 3× with TBST and 1× with TBS followed by incubation in secondary antibody composed of 1:1000 antibody:05% Milk/TBS for 1 h at room temperature. Then the membrane is rinsed 3× with TBST and 1× with TBS and prepared for imaging.

### Western Antibodies

Cell Signaling Technologies (CST) COX IV Mouse mAb 11967s
CST GFP Rabbit Ab 2555s
CST Ikaros Rabbit mAb 14859s
CST Aiolos Rabbit mAb 15103s
CST CK1 Rabbit mAb 2655s
CST eRF3 Rabbit mAb 14980s
MilliporeSigma Flag Mouse mAb F1804-200UG
CST COX IV Mouse mAb 11967s
CST GFP Rabbit Ab 2555s
CST Anti-mouse IgG HRP-linked Antibody 7076s
CST Anti-rabbit IgG HRP-linked Antibody 7074s

### Western Blot Analysis

Using an enhanced chemiluminescence (ECL) kit (Biorad), the PVDF membrane is treated with 1:1 mixture of the reagents and incubated for 5 minutes. Excess liquid is removed from the membrane, which is then immobilized onto a cassette and imaged in a darkroom with film.

Time Course in OVCAR8 eDHFR-Luc Cells

OVCAR8^{eDHR-luc+} cells were plated in black wall/clear bottom (Falcon) 96-well plates (4×10⁴ cells/well) in 200 uL of complete media and incubated with serially diluted compound 7c at −48, −24, −12, −8 and −4 h and analyzed together at 0 h (t=0). Luciferin is prepared to 1× with complete media and 50 uL of Luciferin solution is added to the cells which are then analyzed by plate reader (ThermoFisher Varioskan Plusplate).

### Inhibitor Test

HEK293T^{eDHFR-YFP+} cells in clear (Falcon) 6-well plate (5×10⁵ cells/well) in complete media were incubated with either 500 nM Epoxomicin, 25 μM Hydroxychloriquine HCl, 500 nM MLN4924, or 25 μM 3-Methyladenine for 1 h, followed by the addition of, 100 nM of 7c, 25 μM TMP or 2.5 μM Pomalidomide, where cells were incubated for an additional 12 h. Cells were then isolated as previously described and prepared for Western blot analysis. For readout by flow cytometry, 3×10⁵ HEK293T^{eDHFR-YFP+} cells were seeded in a clear (Falcon) 12-well plate (5×10⁵ cells/well) in complete media and the cells were treated as described above the following day. Cells were washed, trypsinized, and collected following total of 13 h of incubation, and their YFP expression was analyzed on flow cytometer (BD).

### Washout Experiment

HEK293T^{eDHFR-YFP+} cells were seeded in a clear (Falcon) 12-well plate (3×10⁵ cells/well) in complete media. The next day, cells were incubated with 100 nM 7c for 24 h in complete media. Media was removed by vacuum, cells were gently washed with 1 mL PBS 2×, then cell media was replenished. Next, cells were isolated +0 to 24 h after washing and prepared for Western blot analysis.

Jurkat^{eDHFR-YFP+} cells were seeded in a clear (Falcon) 12-well plate (3×10⁵ cells/well) in complete media and incubated overnight. The following day, all wells were dosed with 100 nM of 7c, and cells were sampled at 0, 4, 8, 12, and 24 h following incubation (1 well was sampled per time point). Following 24 h incubation, remaining wells of cells were collected and centrifuged (Thermo Scientific Sorvall Legend X1R) at 1200 rpm for 5 minutes. Cells were washed 3 times with PBS and seeded on a new clear (Falcon) 12-well plate in fresh complete media. The cells were sampled at 3, 6, 24, 48, and 72 h following the drug washout. All cells were fixed in 4% PFA following sampling, and all samples from 10 time points were analyzed together on a flow cytometer (BD).

### General Procedures and Materials

Unless otherwise noted, chemicals were purchased from commercial suppliers at the highest purity grade available and were used without further purification. Thin layer chromatography was performed on 0.25 mm silica gel plates (60F254) using UV light as the visualizing agent. Silica gel (100-200 mesh) was used for column chromatography. Nuclear magnetic resonance spectra were recorded on a 400 MHz spectrometer, and chemical shifts are reported in δ units, parts per million (ppm). Spectra were referenced internally to the residual proton resonance in CDCl3 (δ 7.26 ppm), Methanol-d4 (δ 4.78 ppm), or with tetramethylsilane (TMS, δ 0.00 ppm) as the internal standard. Chemical shifts (δ) were reported as part per million (ppm) on the δ scale downfield from TMS. 13C NMR spectra were referenced to CDCl3 (δ 77.0 ppm, the middle peak) and Methanol-d4 (δ 49.3 ppm). Coupling constants were expressed in Hz. The following abbreviations were used to explain the multiplicities: s=singlet, d=doublet, t=triplet, m=multiplet. High resolution mass spectra were recorded with a micro TOF-Q analyzer spectrometer by using the electrospray mode. Target compounds and/or intermediates were characterized by liquid chromatography/mass spectrometry(LCMS) using a Waters Acquity separation module. Abbreviations used: DCM for dichloromethane, DMF for N,N-dimethylformamide, DMSO for dimethyl sulfoxide, DIPEA for N,N-diisopropylethylamine, MeOH for methanol, NaOH for sodium hydroxide, t-BuOK for potassium tert-butoxide, HBr for hydrobromic acid, ACN for acetonitrile.

### Generation of Stable Cell Lines & Lentivirus Production

Human mesothelioma cell line 145 WT and 145 transduced with human FAP (I45 huFAP) were obtained from the Albelda laboratory at the University of Pennsylvania. Both the 145 WT and 145 huFAP cells were further transduced to express luciferase with pTRPE lentiviral vector encoding firefly luciferase-T2A-mCherry. Lentivirus was packaged in HEK293T/17 (ATCC) by transfecting the cells with pTRPE luciferase-T2A-mCherry construct and 2^{nd} generation packaging plasmids (psPAX and pMD2) at a ratio of 4:3:2 by mass. A full media change was performed on cells 24 hours post-transfection, and the supernatants containing lentiviral particles were collected at the 48 hour timepoint. Collected supernatants were centrifuged for 10 minutes at 1200 rpm to remove any cell debris and filtered through a 0.45 μm filter (Millipore Sigma). Lentiviral particles were concentrated using a 100-kDa centrifugal filter concentrator (Millipore Sigma). 145 WT and 145 huFAP were transduced with the concentrated lentiviral particles overnight in presence of 8 μg/mL of polybrene, washed and incubated with fresh media for 1-2 days, and passaged. Following expansion, the cells were sorted on mCherry expression through fluorescence-activated cell sorting (BD Biosciences) to generate stable 145 WT and 145 huFAP cells expressing luciferase (I45 WT-Luc and 145 huFAP-Luc).

### Cell Lines

Human mesothelioma cell line 145 WT-Luc and 145 huFAP-Luc were maintained in RPMI 1640 supplemented with 10% fetal bovine serum (FBS), 100 U/mL penicillin, and 100 g/mL streptomycin sulfate. All reagents from ThermoFisher Scientific. Cells were maintained in a humidified incubator at 37° C.

### Cloning of CAR Constructs

pTRPE lentiviral vector encoding FAP-scFv (4G5)-CD8 hinge-4-1BB-CD3z was obtained from the Albelda and Pure laboratories at the University of Pennsylvania. The CAR targets both human and murine FAP-expressing cells. A gBlock of CD8 hinge-4-1BB-CD3z-eDHFR was ordered and cloned downstream of the FAP-scFv to make a pTRPE FAP CAR-eDHFR direct fusion (DF) construct. A T2A-TagBFP gene was further cloned downstream of the eDHFR (pTRPE FAP CAR-eDHFR DF-T2A-BFP) later in the course of the project to help with the assessment of transduction, flow-based sorting of CAR⁺ T cells, and in vivo animal experiments.

### Primary Human CAR T Cell Generation

Primary human T cells collected from healthy volunteers were obtained from the Human Immunology Core at the University of Pennsylvania. All human specimens were collected under a University Institutional Review Board's approved protocols following informed consent from the volunteers. CD4⁺ and CD8⁺ T cells were mixed at a 1:1 ratio and activated by incubating with anti-CD3/anti-CD28 antibody-coated magnetic beads (Dynabeads, Thermo Fisher Scientific) at a ratio of 3:1 beads to T cells. Following 16 hours of incubation with the beads, either pTRPE FAP CAR-eDHFR DF or pTRPE FAP CAR-eDHFR DF-T2A-BFP lentivirus (generated as described under “Generation of Stable Cell Lines & Lentivirus Production”) was added to the activated T cells at an MOI of 5-8. The T cells were expanded for 10 days before characterization and cell sorting.

### Flow Cytometry

Generated CAR T cells were pelleted, resuspended in 2% BSA in PBS (Invitrogen), and incubated with Alexa Fluor® 647 AffiniPure F(ab′2) fragment goat anti-mouse IgG (Jackson ImmunoResearch Laboratories) for 30 minutes at room temperature. Stained cells were washed 3 times with PBS, and were analyzed on a flow cytometer (LSR II, BD Biosciences) for CAR and/or BFP expression. All flow data were analyzed using FlowJo software (FlowJo).

### Dose Response Assay

2×10⁵ primary human FAP-eDHFR DF CAR T cells were incubated with different concentrations of TMP-POM 7c for 4 and 24 hours in a 96-well plate. Following incubation, the cells were washed and stained with Alexa Fluor® 647 AffiniPure F(ab′2) fragment goat anti-mouse IgG (Jackson ImmunoResearch Laboratories) as described under “Flow Cytometry”, and their surface expression was analyzed on a flow cytometer (LSR II, BD Biosciences).

### Washout Kinetic Assay

6×10⁵ primary human FAP-eDHFR DF CAR T cells were incubated with 100 nM of TMP-POM 7c or vehicle control (DMSO) in a 12-well plate, and cells were sampled at different time points (0, 4, 8, and 24 hours following incubation) to assess for their CAR expression by flow cytometry. Following a 24 hour incubation with TMP-POM 7c, the remaining cells were washed multiple times with PBG (Corning) to remove the drug, returned to culture in a new 12-well plate, and then again sampled serially at different time points following the wash (3, 6, 24, 48, and 72 hours post-washout). All cells were stained for CAR and fixed in 4% following sampling, and were analyzed on a flow cytometer (LSR II, BD Biosciences).

### Cytotoxicity and Cytokine Release Assay

1×10⁴ of 145 WT-Luc and 145 huFAP-Luc target cells were seeded into a 96-well plate. The following day, either non-transduced (NTD)—but activated—control T cells or effector FAP-eDHFR DF CAR T cells that were pre-incubated with various doses of TMP-POM 7c were added to the target cells at a range of effector-to-target (E:T) ratios from 5:1 to 20:1. Following an overnight co-incubation of T cells and target cells, supernatants were collected to quantify IFγ and TNFα concentration by ELISA (Abcam), and the target cell viability was assessed by adding D-luciferin (GoldBio) to the sample wells to a final concentration of 0.15 mg/mL. Following a 5 minute incubation of cells with D-luciferin, the plate was read on a plate reader (Thermo Fisher Scientific) with 500 ms integration time.

### Inhibitor Test

5×10⁵ primary human FAP-eDHFR DF CAR T cells were seeded in a 12-well plate and pre-incubated with 50 nM bafilomycin (Sigma), 100 nM epoxomicin (Selleckchem), 1 uM MG132 (Sigma), or 500 nM MLN4924 (Pevonedistat; Selleckchem) for 1 hour. Following pre-incubation, either 100 nM 7c or vehicle control DMSO (Sigma) was added to the wells, and cells were incubated with the drug for another 4 hours. Following incubation, the cells were washed and their surface CAR expression was assessed on a flow cytometer (BD Biosciences).

In Vitro Cell Uptake with [¹⁸F]Fluoropropyl-Trimethoprim (FPTMP)

3×10⁶ CAR T cells were incubated with 6×10⁶ counts per minute (cpm) of [¹⁸F]FPTMP for 60 minutes in the presence or absence of unlabeled 5 μM TMP. Following incubation, cells were centrifuged at 1200 rpm and washed 3 times with cold PBS (Corning). After the third wash, the cell pellet was resuspended in 900 μL of PBS and split into 3 technical replicates of 300 μL. Radiotracer uptake was quantified on a gamma counter (PerkinElmer) and analyzed by dividing counts by the injected dose (ID) of [¹⁸F]FPTMP. The final uptake was reported as a ratio between % ID normalized per 10⁶ cells (% ID/10⁶ cells) of the [¹⁸F]FPTMP group and the blocked control (i.e. cells that were incubated with both unlabeled TMP and [¹⁸F]FPTMP).

### Scheme 1

The general synthesis of TMP-Protac^{a} compounds described herein is shown in Scheme 1. The reaction conditions for steps (i), (ii), (iii), (iv), (v) and (vi) is as follows:
(i) HBr, 90° C., 20 min, 1M NaOH;
(ii) t-BuOK, DMSO, 2 h/Cs₂CO₃, DMF, 70° C., 12 hrs;
(iii) K₂CO₃, MeOH, H₂O, 70° C., 12 hrs;
(iv) DIPEA, DMF, 90° C., 12 hrs;
(v) TFA, DCM, rt, overnight;
(vi) Cs₂CO₃, iodomethane, DMF; and
(vii) PyAOP, DIPEA, DMF, 30 min, rt.

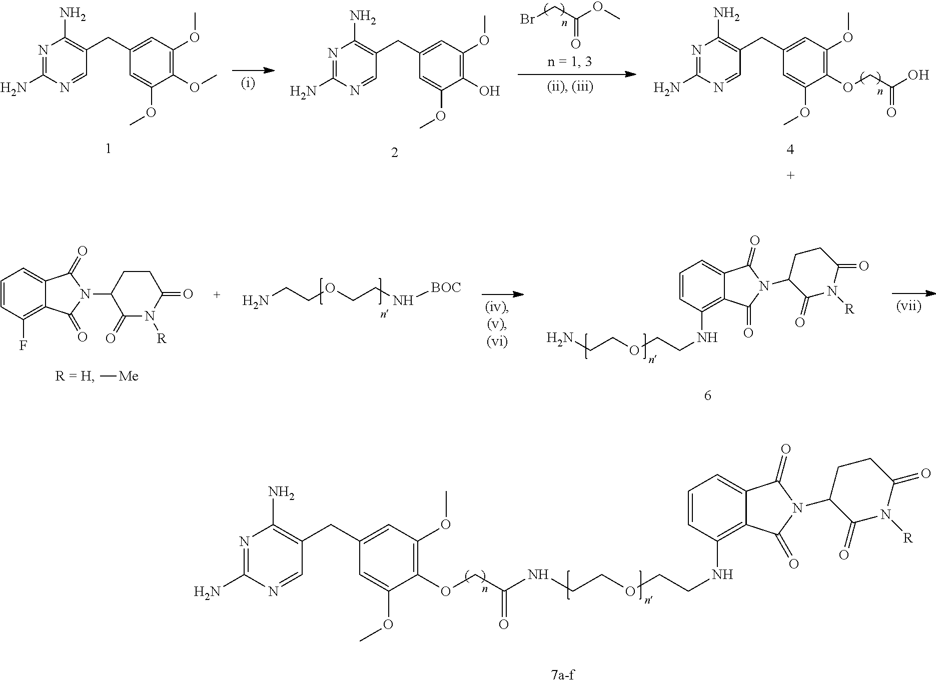

### Example 1—Synthesis of Compound 7a

### Synthesis of 4-((2,4-diaminopyrimidin-5-yl)methyl)-2,6-dimethoxyphenol (2)

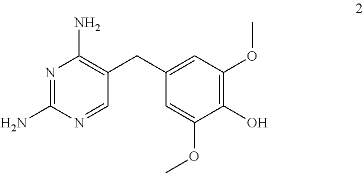 [SMILES: COc1cc(Cc2cnc(N)nc2N)cc(OC)c1O]

Dissolve trimethoprim (5.00 g, 17.12 mmol) in HIBr (62 mL, 48% in H₂O) and stir at 95° C. for 30 minutes and then quenched by slow addition of 12 mL 50% NaOH. Reaction mixture was allowed to cool to room temperature, and placed at 4° C. overnight, allowing crystals to form. Filter the precipitate and wash with ice cold water. Dissolve the collected precipitate in boiling H₂O, add 1 N NaOH to neutralize, leading to recrystallization. Crystals are washed with water and filtered under vacuum, to afford 4-((2,4-diaminopyrimidin-5-yl)methyl)-2,6-dimethoxyphenol 2 (3.2 g, 68%) as a white solid. LCMS(ESI); m/z: [M+H]⁺ calcd. for C13H17N4O3, 277.13; Found 277.35. See, e.g., Mark A. Sellmyer, 1, Iljung Leea, Catherine Houa, Chi-Chang Wenga, Shihong Lia, Brian P. Liebermana, Chenbo Zenga, David A. Mankoffa, and Robert H. Mach PNAS, 2017,8372-8377.

### Synthesis of methyl 2-(4-((2,4-diaminopyrimidin-5-yl)methyl)-2,6-dimethoxyphenoxy)acetate (3a)

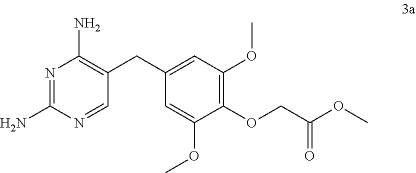 [SMILES: COC(=O)COc1c(OC)cc(Cc2cnc(N)nc2N)cc1OC]

To a solution of 2 (1.2 g, 4.3 mmol) in anhydrous DMSO (25 mL) was added t-BuOK (530 mg, 4.7 mmol, 1.1 equiv.) with stirring under Ar atmosphere. The solution was stirred at room temperature for few minutes, turned to deep orange. To the solution add methyl bromoacetate (0.724 mg, 4.7 mmol, 1.1 equiv.), and the reaction was stirred at room temperature for 2 h. The reaction was monitored by TLC (10% MeOH/DCM) and after reaction completion, solvent was removed under reduced pressure, and the residual brown oil was subjected to column chromatography on silica gel with elution with 5-10% CH₂Cl₂/CH₃OH to afford 3a (700 mg, 47%) as a whitish-brown solid. LCMS(ESI); m/z: [M+H]⁺ calcd. for C16H21N4O5, 349.15; Found 349.28. See, e.g., Wei Liu et al. J. Am. Chem. Soc. 2014, 136, 4468-4471.

### Synthesis of methyl 4-(4-((2,4-diaminopyrimidin-5-yl)methyl)-2,6-dimethoxyphenoxy)butanoate (3b)

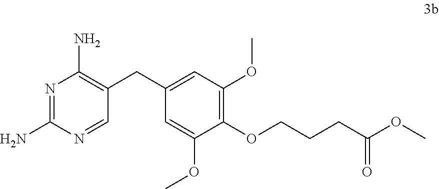 [SMILES: COC(=O)CCCOc1c(OC)cc(Cc2cnc(N)nc2N)cc1OC]

To the solution of 2 (1.2 g, 4.3 mmol) in anhydrous DMF (40 mL), cesium carbonate (2.8 g, 8.6 mmol, 2 equiv.) was added. The mixture was allowed to stir at room temperature for few minutes, the color changed to deep orange. To the solution was added methyl 4-bromo butanoate (0.778 g, 4.3 mmol), and the reaction was stirred at 70° C. overnight. The reaction completion was monitored by TLC (10% Methanol/DCM). DMF was removed under high vacuum. To the residue add water and extracted with ethyl acetate (2×50 mL). The organic layer was washed with aqueous sodium bicarbonate solution, dried over sodium sulfate, and the solvent was removed under reduced pressure and trituration with isopropyl ether afforded 3b (1.1 g, 69%) as a light brown solid. LCMS(ESI); m/z: [M+H]⁺ calcd. for C18H25N4O5, 377.18; Found 377.44.

### Synthesis of 2-(4-((2,4-diaminopyrimidin-5-yl)methyl)-2,6-dimethoxyphenoxy)acetic acid (4a)

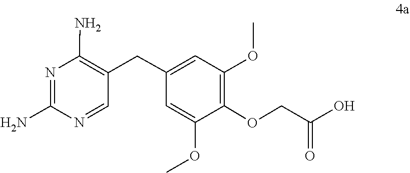 [SMILES: COc1cc(Cc2cnc(N)nc2N)cc(OC)c1OCC(=O)O]

To the solution of methyl 2-(4-((2,4-diaminopyrimidin-5-yl)methyl)-2,6-dimethoxyphenoxy)acetate 3a (491 mg, 1.41 mmol) in methanol (10 mL), added potassium carbonate (876 mg, 6.3 mmol, 4.5 equiv.), followed by water (4 mL) and the reaction was heated at 60° C. overnight. Evaporate methanol under reduced pressure, add water (25 mL) to the residue and neutrals were removed by extraction with ethyl acetate (2×50 mL). Aqueous layer was neutralized with 6 Molar HCl to pH ˜7. It was concentrated to ˜10-15 mL, put in the fridge overnight. Filtration afforded 4a (332 mg, 71%) as light brown solid which was quite pure for next step. LCMS(ESI); m/z: [M+H]⁺ calcd. for C15H19N4O5, 335.14; Found 335.34.

### Synthesis of 4-(4-((2,4-diaminopyrimidin-5-yl)methyl)-2,6-dimethoxyphenoxy)butanoic acid (4b)

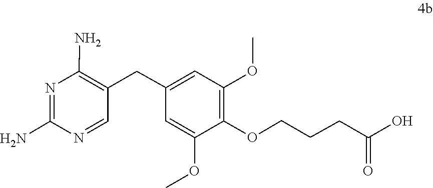 [SMILES: COc1cc(Cc2cnc(N)nc2N)cc(OC)c1OCCCC(=O)O]

To the solution of methyl 4-(4-((2,4-diaminopyrimidin-5-yl)methyl)-2,6-dimethoxyphenoxy)butanoate 3b (1 g, 2.7 mmol) in methanol (18 mL), added potassium carbonate (1.7 g, 12 mmol, 4.5 equiv.), followed by water (6 mL) and the reaction was heated at 60° C. overnight. Evaporate methanol under reduced pressure, add water (25 ml) to the residue and neutrals were removed by extraction with ethyl acetate (2×50 mL). Aqueous layer was neutralized with 6 Molar HCl to pH ˜7. It was concentrated to ˜10-15 mL, put in the fridge overnight. Filtration afforded 4b (726 mg, 74%) as light brown solid which was quite pure for next step. LCMS(ESI); m/z: [M+H]⁺ calcd. for C17H23N4O5, 363.17 Found 363.34.

### Synthesis of tert-butyl (2-(2-((2-(2,6-dioxopiperidin-3-yl)-1,3-dioxoisoindolin-4-yl)amino)ethoxy)ethyl)carbamate (5a)

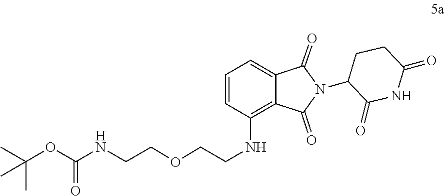 [SMILES: CC(C)(C)OC(=O)NCCOCCNc1cccc2c1C(=O)N(C1CCC(=O)NC1=O)C2=O]

To the solution of 2-(2,6-dioxopiperidin-3-yl)-4-fluoroisoindoline-1,3-dione (276 mg, 1 mmol) in DMF (3 ml) add N-Boc-2-(2-Aminoethoxy)ethanamine (306 mg, 1.5 mmol) followed by DIPEA (0.7 mL, 4 mmol, 4 equiv.) and the reaction mixture was heated at 90° C. for 12 h. After the reaction completion monitored by the TLC, the dark green reaction mixture was poured into water (10 mL) and extracted with ethyl acetate(2×10 mL). The combined organic layer was washed with brine, dried over sodium sulfate, and the solvent was removed under reduced pressure. The residue was purified by column chromatography to afford 5a in (262 mg, 57%) as yellow-green syrup. See, e.g., Ganna Posternakiet et al. Nat. Chem. Biol. 2020, 16, 1170-1178.

### Synthesis of tert-butyl (2-(2-(2-((2-(2,6-dioxopiperidin-3-yl)-1,3-dioxoisoindolin-4-yl)amino)ethoxy)ethoxy)ethyl)carbamate (5b)

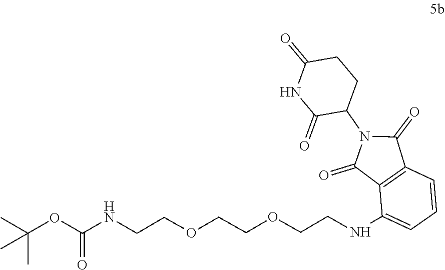 [SMILES: CC(C)(C)OC(=O)NCCOCCOCCNc1cccc2c1C(=O)N(C1CCC(=O)NC1=O)C2=O]

The procedure analogous to that described for compound 5a (276 mg, 1 mmol) and tert-butyl (2-(2-(2-aminoethoxy)ethoxy)ethyl)carbamate (372 mg, 1.5 mmol) as starting materials afforded 5b (300 mg, 60%) as yellow-green syrup.

### Synthesis of 4-((2-(2-aminoethoxy)ethyl)amino)-2-(2,6-dioxopiperidin-3-yl)isoindoline-1,3-dione (6a)

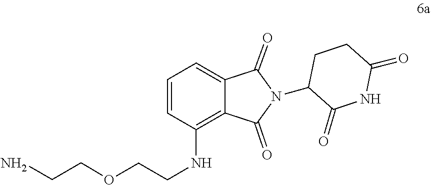 [SMILES: NCCOCCNc1cccc2c1C(=O)N(C1CCC(=O)NC1=O)C2=O]

To the solution of 5a (262 mg, 0.57 mmol) in DCM (5 mL), was added TFA(1.1 mL, 25 equiv.) and the reaction mixture was stirred at room temperature for 5 h. After reaction completion monitored by TLC, DCM (20 mL) was added and the organic layer was washed with sodium carbonate solution, dried over sodium sulfate, and solvent was removed under reduced pressure to give 6a (174 mg, 85%) as yellow solid. LCMS(ESI); m/z: [M+H]⁺ calcd. for C17H21N4O5, 361.15; Found 361.32.

### Synthesis of 4-((2-(2-(2-aminoethoxy)ethoxy)ethyl)amino)-2-(2,6-dioxopiperidin-3-yl)isoindoline-1,3-dione (6b)

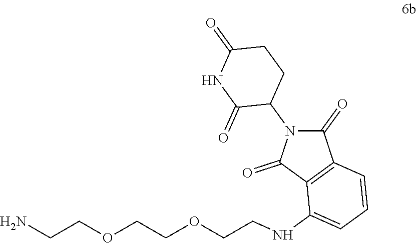 [SMILES: NCCOCCOCCNc1cccc2c1C(=O)N(C1CCC(=O)NC1=O)C2=O]

The procedure analogous to that described for compound 6a, using 5b (287 mg, 0.57 mmol) as starting material to afford 6b (200 mg, 87% yield) as yellow solid. LCMS(ESI); m/z: [M+H]⁺ calcd. for C19H25N4O6, 405.18; Found 405.44.

### Synthesis of 2-(4-((2,4-diaminopyrimidin-5-yl)methyl)-2,6-dimethoxyphenoxy)-N-(2-(2-(2-((2-(2,6-dioxopiperidin-3-yl)-1,3-dioxoisoindolin-4-yl)amino)ethoxy)ethoxy)ethyl)acetamide (7a)

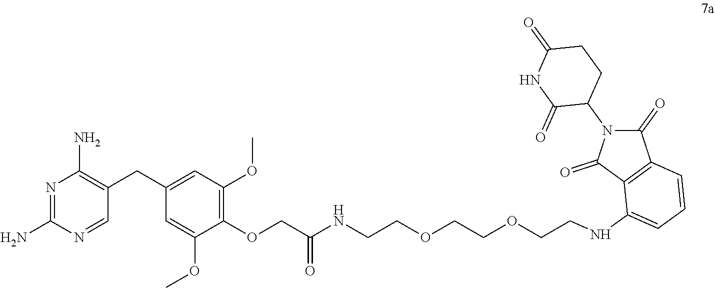 [SMILES: COc1cc(Cc2cnc(N)nc2N)cc(OC)c1OCC(=O)NCCOCCOCCNc1cccc2c1C(=O)N(C1CCC(=O)NC1=O)C2=O]

To the solution of 2-(4-((2,4-diaminopyrimidin-5-yl)methyl)-2,6-dimethoxyphenoxy)acetic acid 4a (50 mg, 0.15 mmol) in DMF (2 mL) was added 4-((2-(2-(2-aminoethoxy)ethoxy)ethyl)amino)-2-(2,6-dioxopiperidin-3-yl)isoindoline-1,3-dione 6b (61 mg, 0.15 mmol) followed by DIPEA (0.2 mL, 0.11 mmol, 7.5 equiv.) and PyAOP(98 mg, 0.19 mmol, 1.25 equiv.) and the reaction mixture was stirred at room temperature for half an hour. After reaction completion as monitored by the TLC, it was poured into water and extracted with DCM (2×10 mL). The combined organic layer was washed with water and brine, dried over sodium sulfate, and solvent was removed under reduced pressure. Column chromatography was performed to isolate the product 7a (31 mg, 29%) as yellow solid. ¹H NMR (400 MHz, CDCl₃) δ: 12.27 (s, 1H), 8.04-8.03 (m, 1H), 7.79 (s, 1H), 7.49-7.45 (m, 1H), 7.09 (d, J=7.2 Hz, 1H), 6.87 (d, J=8.8 Hz, 1H), 6.43 (t, J=5.6 Hz, 1H), 6.35 (s, 2H), 5.66 (s, 2H), 5.29 (s, 1H), 4.94-4.90 (m, 1H), 4.54-4.44 (m, 2H), 3.79 (s, 5H), 3.73-3.42 (m, 13H), 2.89-2.72 (m, 3H), 2.16-2.11 (m, 1H) ppm. ¹³C NMR (100 MHz, CDCl₃) δ: 173.5, 170.7, 170.1, 169.4, 167.7, 163.1, 152.6, 146.8, 136.1, 135.5, 134.6, 132.5, 116.7, 111.7, 110.3, 105.8, 104.7, 72.8, 70.5, 70.2, 70.0, 69.4, 56.1, 48.9, 42.5, 38.9, 34.6, 31.6, 22.8 ppm. LCMS(ESI); m/z: [M+H]⁺ calcd. for C34H41N8O10, 721.29; Found 721.42. HRMS calcd for C34H41N8O10 [M+H⁺], 721.2938; found, 721.2946.

### Example 2—Synthesis of Compound 7b

### Synthesis of 4-(4-((2,4-diaminopyrimidin-5-yl)methyl)-2,6-dimethoxyphenoxy)-N-(2-(2-((2-(2,6-dioxopiperidin-3-yl)-1,3-dioxoisoindolin-4-yl)amino)ethoxy)ethyl)butanamide 7b

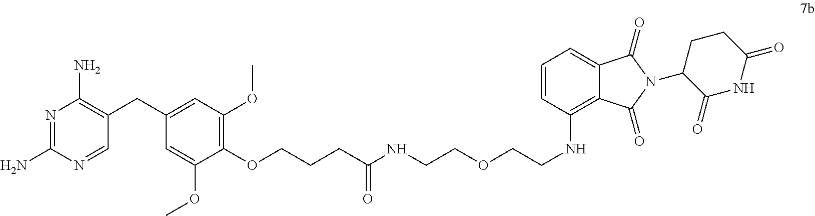 [SMILES: COc1cc(Cc2cnc(N)nc2N)cc(OC)c1OCCCC(=O)NCCOCCNc1cccc2c1C(=O)N(C1CCC(=O)NC1=O)C2=O]

The procedure analogous to that described for compound 7a, with 4-(4-((2,4-diaminopyrimidin-5-yl)methyl)-2,6-dimethoxyphenoxy)butanoic acid 4b (51 mg, 0.14 mmol) and 4-((2-(2-aminoethoxy)ethyl)amino)-2-(2,6-dioxopiperidin-3-yl)isoindoline-1,3-dione 6a (51 mg, 0.14 mmol) as starting materials furnished 7b (38 mg, 38%) as yellow solid. ¹H NMR (400 MHz, CDCl₃) δ: 11.07 (s, 1H), 7.53-7.49 (m, 2H), 7.33 (s, 1H), 7.10 (d, J=7.2 Hz, 1H), 6.87 (d, J=8.4 Hz, 1H), 6.67-6.55 (m, 2H), 6.31 (s, 2H), 5.77 (s, 1H), 5.02-5.00 (m, 1H), 3.99 (t, J=5.6 Hz, 2H), 3.77 (s, 6H), 3.71-3.66 (m, 2H), 3.58-3.55 (m, 4H), 3.43-3.36 (m, 3H), 2.81-2.73 (m, 3H), 2.53 (t, J=6.8 Hz, 2H), 2.11-2.04 (m, 3H) ppm. ¹³C NMR (100 MHz, CDCl₃) δ: 173.6, 173.4, 170.9, 169.5, 167.6, 163.7, 153.7, 146.8, 136.3, 135.7, 132.4, 116.9, 111.8, 110.3, 105.2, 72.3, 70.2, 68.5, 56.0, 48.6, 42.1, 39.1, 34.2, 33.2, 31.1, 26.2, 22.7 ppm. LCMS(ESI); m/z: [M+H]⁺ calcd. For C34H41N8O9, 705.30; Found 705.44. HRMS calcd for C34H41N8O9 [M+H⁺], 705.2997; found, 705.2980.

### Example 3—Synthesis of Compound 7c

### Synthesis of 4-(4-((2,4-diaminopyrimidin-5-yl)methyl)-2,6-dimethoxyphenoxy)-N-(2-(2-(2-((2-(2,6-dioxopiperidin-3-yl)-1,3-dioxoisoindolin-4-yl)amino)ethoxy)ethoxy)ethyl)butanamide 7c

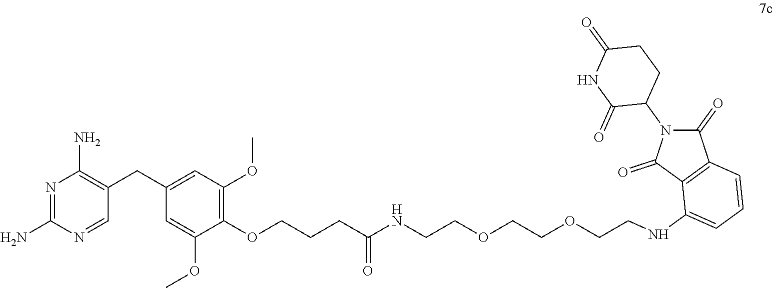 [SMILES: COc1cc(Cc2cnc(N)nc2N)cc(OC)c1OCCCC(=O)NCCOCCOCCNc1cccc2c1C(=O)N(C1CCC(=O)NC1=O)C2=O]

The procedure analogous to that described for compound 7a, with 4-(4-((2,4-diaminopyrimidin-5-yl)methyl)-2,6-dimethoxyphenoxy)butanoic acid 4b (51 mg, 0.14 mmol) and 4-((2-(2-(2-aminoethoxy)ethoxy)ethyl)amino)-2-(2,6-dioxopiperidin-3-yl)isoindoline-1,3-dione 6b (57 mg, 0.14 mmol) as starting materials furnished 7c (46 mg, 44%) as yellow solid. ¹H NMR (400 MHz, CDCl₃) δ: 12.39 (s, 1H), 7.78 (s, 1H), 7.47 (t, J=7.6 Hz, 1H), 7.09 (d, J=7.2 Hz, 1H), 6.88 (d, J=8.4 Hz, 1H), 6.46 (t, J=5.2 Hz, 2H), 6.34 (s, 2H), 5.56 (s, 1H), 5.28 (s, 1H), 4.94-4.90 (m, 1H), 3.95 (t, J=4.2 Hz, 2H), 3.76 (s, 6H), 3.71-3.59 (m, 8H), 3.52-3.40 (m, 6H), 2.88-2.74 (m, 3H), 2.45 (t, J=7.2 Hz, 2H), 2.13-1.98 (m, 4H) ppm. ¹³C NMR (100 MHz, CDCl₃) δ: 173.5, 173.2, 170.8, 169.4, 167.6, 163.0, 162.3, 156.3, 153.6, 146.8, 136.1, 135.6, 134.1, 132.5, 116.7, 111.7, 110.3, 105.9, 105.0, 72.0, 70.5, 70.1, 69.9, 69.3, 56.1, 48.9, 42.4, 39.2, 34.8, 33.3, 31.6, 26.2, 22.7 ppm. LCMS(ESI); m/z: [M+2H]⁺ calcd. for C36H46N8O10, 750.33 Found 750.46. HRMS calcd for C36H45N8O10 [M+H]⁺, 749.3259; found, 749.3257.

### Example 4—Synthesis of Compound 7e and 7f

### 4-(4-((2,4-diaminopyrimidin-5-yl)methyl)-2,6-dimethoxyphenoxy)-N-(20-((2-(2,6-dioxopiperidin-3-yl)-1,3-dioxoisoindolin-4-yl)amino)-3,6,9,12,15,18-hexaoxaicosyl)butanamide (7e)

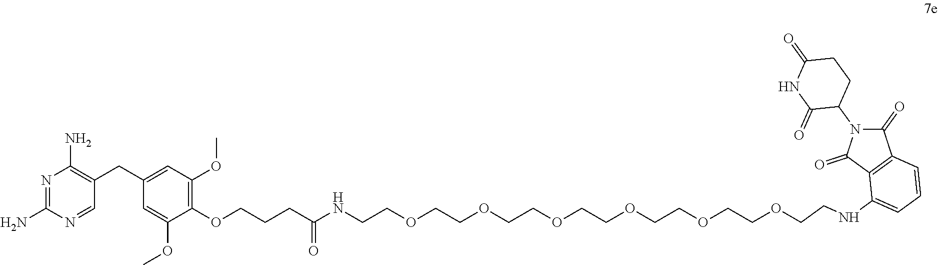 [SMILES: COc1cc(Cc2cnc(N)nc2N)cc(OC)c1OCCCC(=O)NCCOCCOCCOCCOCCOCCOCCNc1cccc2c1C(=O)N(C1CCC(=O)NC1=O)C2=O]

The procedure analogous to that described for compound 7a, with 4-(4-((2,4-diaminopyrimidin-5-yl)methyl)-2,6-dimethoxyphenoxy)butanoic acid 4b (51 mg, 0.14 mmol) and 4-((20-amino-3,6,9,12,15,18-hexaoxaicosyl)amino)-2-(2,6-dioxopiperidin-3-yl)isoindoline-1,3-dione 6c (81.2 mg, 0.14 mmol) as starting material furnished 7e (34 mg, 26%) as yellow syrup. ¹H NMR (400 MHz, MeOD-d4) δ:7.59-7.53 (m, 2H), 7.12-7.08 (m, 2H), 6.56 (s, 2H), 5.11-5.06 (m, 1H), 3.94 (t, J=6.0 Hz, 2H), 3.82 (s, 6H), 3.74 (t, J=5.2 Hz, 2H), 3.67-3.51 (m, 24H), 3.40-3.34 (m, 7H), 2.90-2.76 (m, 1H), 2.50-2.46 (m, 2H), 2.02-1.95 (m, 2H). ¹³C NMR (100 MHz, MeOD-d4) δ: 176.3, 175.1, 172.0, 171.0, 169.6, 165.0, 162.5, 155.2, 154.1, 148.5, 137.5, 137.0, 136.3, 134.2, 118.6, 112.3, 108.8, 107.2, 73.6, 72.0, 71.9, 71.8, 71.5, 70.9, 56.9, 43.6, 40.8, 34.7, 33.9, 32.5, 27.7, 24.0 ppm. LCMS(ESI); m/z: [M+H]⁺ calcd. for C44H61N8O14, 925.43; Found 925.64. HRMS calcd for C44H61N8O14 [M+H]⁺, 925.4307; Found 925.4298.

### 4-(4-((2,4-diaminopyrimidin-5-yl)methyl)-2,6-dimethoxyphenoxy)-N-(2-(2-(2-((2-(1-methyl-2,6-dioxopiperidin-3-yl)-1,3-dioxoisoindolin-4-yl)amino)ethoxy)ethoxy)ethyl)butanamide (7f)

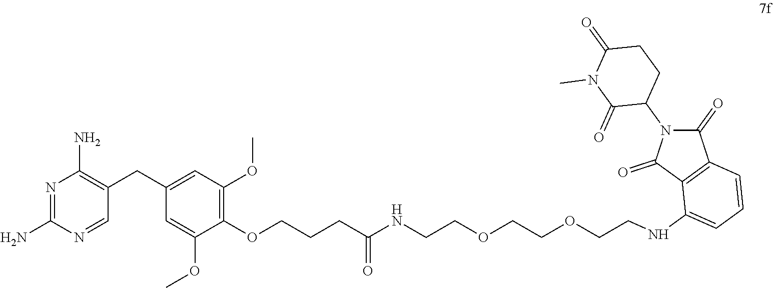 [SMILES: COc1cc(Cc2cnc(N)nc2N)cc(OC)c1OCCCC(=O)NCCOCCOCCNc1cccc2c1C(=O)N(C1CCC(=O)N(C)C1=O)C2=O]

The procedure analogous to that described for compound 7a, with 4-(4-((2,4-diaminopyrimidin-5-yl)methyl)-2,6-dimethoxyphenoxy)butanoic acid 4b (18 mg, 0.05 mmol) and 4-((2-(2-(2-aminoethoxy)ethoxy)ethyl)amino)-2-(1-methyl-2,6-dioxopiperidin-3-yl)isoindoline-1,3-dione (21 mg, 0.05 mmol) as starting material furnished 7f (12 mg, 31%) as yellow solid. ¹H NMR (400 MHz, CDCl₃) δ: 8.03 (s, 1H), 7.47-7.43 (m, 1H), 7.24 (s, 1H), 7.05 (d, J=7.2 Hz, 1H), 6.87 (d, J=8.8 Hz, 1H), 6.81-6.78 (m, 1H), 6.34 (s, 2H), 4.93-4.88 (m, 1H), 3.97-3.94 (m, 2H), 3.77 (s, 6H), 3.70-3.65 (m, 3H), 3.59-3.52 (m, 7H), 3.44-3.42 (m, 3H), 3.15 (s, 3H), 2.99 (s, 1H), 2.90 (s, 1H), 2.75-2.70 (m, 2H), 2.53-2.49 (m, 2H), 2.09-1.98 (m, 4H). LCMS(ESI); m/z: [M+2H]⁺ calcd. for C37H48N8O14, 764.35; Found 764.85. HRMS calcd for C37H47N8O10 [M+H]⁺, 763.3410; Found 763.3415.

### Example 5—Time and Dose-Dependent Degradation of YFP in Jurkat-DYL

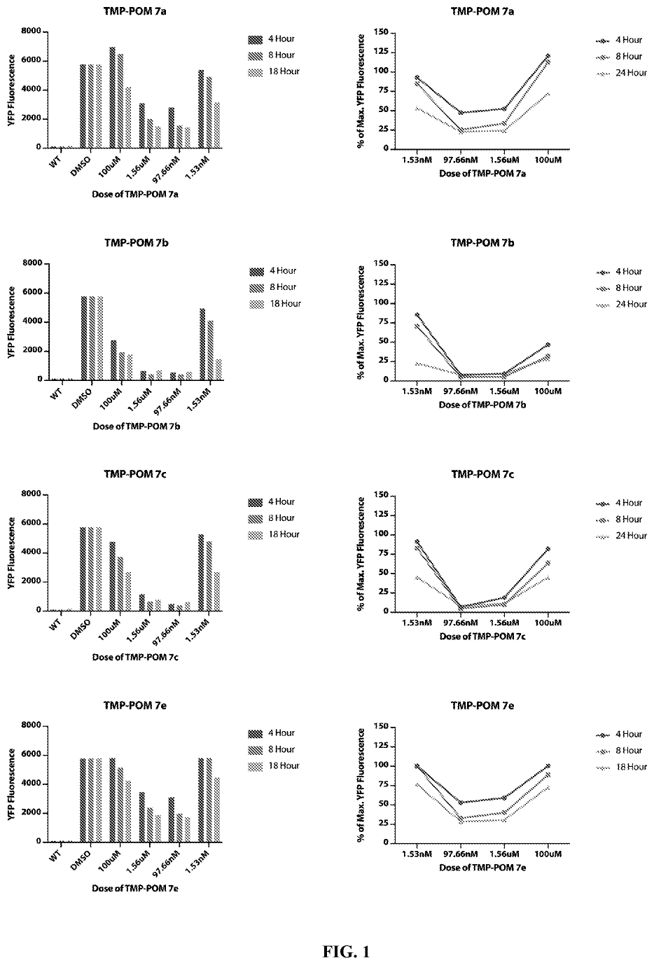

FIG. 1 demonstrates the time and dose dependency of degradation of YFP in Jurkat-DYL using compounds 7a, 7b, 7c and 7e.

FIG. 1A shows the YFP fluorescence of Jurkat WT and Jurkat-DYL measured using flow cytometry following 4, 8, and 18-hour of incubation with DMSO or varying doses of compounds 7a, 7b, 7c and 7e.

FIG. 1B shows similar data as presented as in FIG. 1A but represented as % of maximum YFP expression normalized to Jurkat-DYL treated with DMSO.

As can be seen, compounds 7a, 7b, 7c and 7e degrade eDHFR fusion proteins to low/no functional expression. The degradation allows for fine-tuning of intended therapeutic responses or abrogating an unintended toxic response.

### Example 6—Degradation of eDHFR-POI

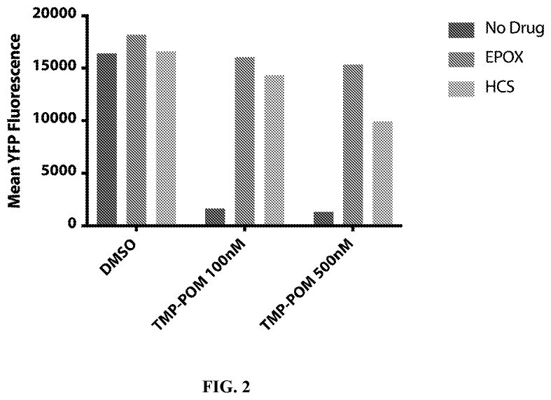

FIG. 2 demonstrates degradation of eDHFR-POI using compounds 7a, 7b, 7c and 7e. In FIG. 2, flow cytometric analysis is used to measure Jurkat-DYL cells expressing DHFR-YFP (DY) direct fusion protein pre-treated with DMSO, epoxomicin (EPOX), or hydroxychloroquine sulfate (HCS) for 2 hours before DMSO or compound 7b treatment for 4 hours.

As can be seen, eDHFR-POI is degraded by compound 7b primarily via a proteosome-mediated mechanism of degradation.

### Example 7—Degradation of Luciferase

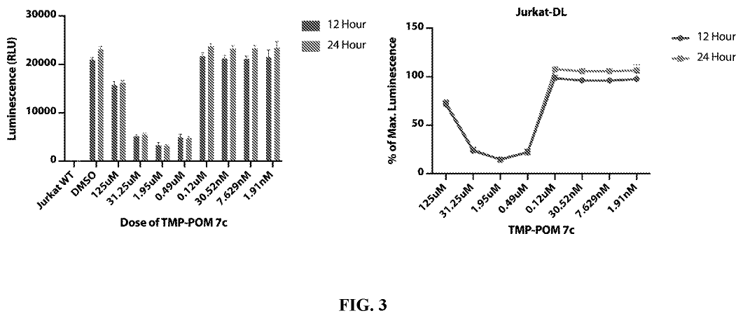

FIG. 3 demonstrates degradation of luciferase using eDHFR/compound 7c.

FIG. 3A shows luminescence from Jurkat WT and Jurkat-DL is measured following 4, 8, and 18-hour of incubation with DMSO or varying doses of compound 7c. Data shown are in triplicates, error bars=SD.

FIG. 3B shows similar data is presented as in FIG. 3A but represented as % of maximum luminescence output normalized to Jurkat-DL treated with DMSO. Data shown are in triplicates, error bars=SD.

As can be seen, luciferase is degraded by eDHFR/compound 7c.

### Example 8—Time and Dose-Dependent Degradation of YFP and Luciferase in Human Embryonic Kidney HEK293/17 Cells

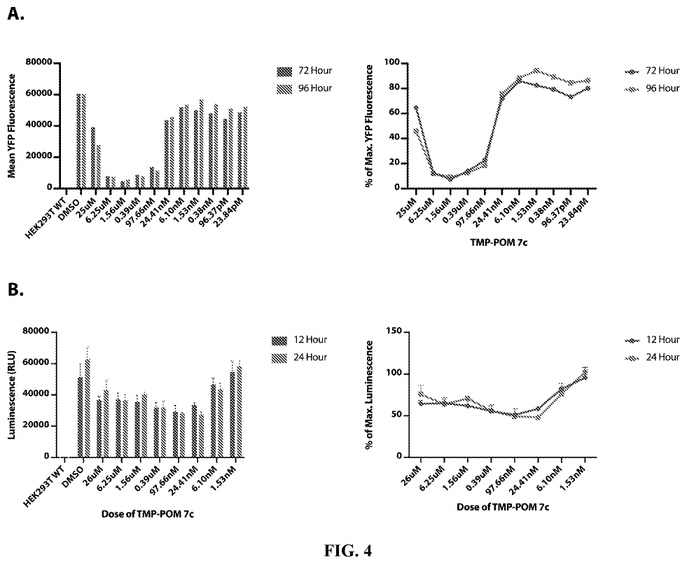

FIG. 4 demonstrates the time and dose dependency of degradation of YFP and Luciferase in human embryonic kidney HEK293T/17 cells using compound 7c.

FIG. 4A shows YFP fluorescence of HEK293T WT and HEK293T-DYL are measured using flow cytometry following 72 and 96 hours of incubation with DMSO or varying doses of compound 7c. The left panel represents the mean YFP fluorescence and the right panel represents the same data in percent of maximum YFP fluorescence normalized to HEK293T-DYL treated with DMSO. Data shown are in triplicates, error bars=SD.

FIG. 4B shows luminescence from HEK293T WT and HEK293T-DL are measured following 12 and 24-hours of incubation with DMSO or varying doses of compound 7c. The left panel represents the mean YFP fluorescence and the right panel represents the same data in percent of maximum YFP fluorescence normalized to HEK293T-DYL treated with DMSO. Data shown are in triplicates, error bars=SD.

As can be seen, YFP and luciferase are degraded by compound 7c.

### Example 9—Degradation of CAR Molecules From the Surface of CAR-T Cells

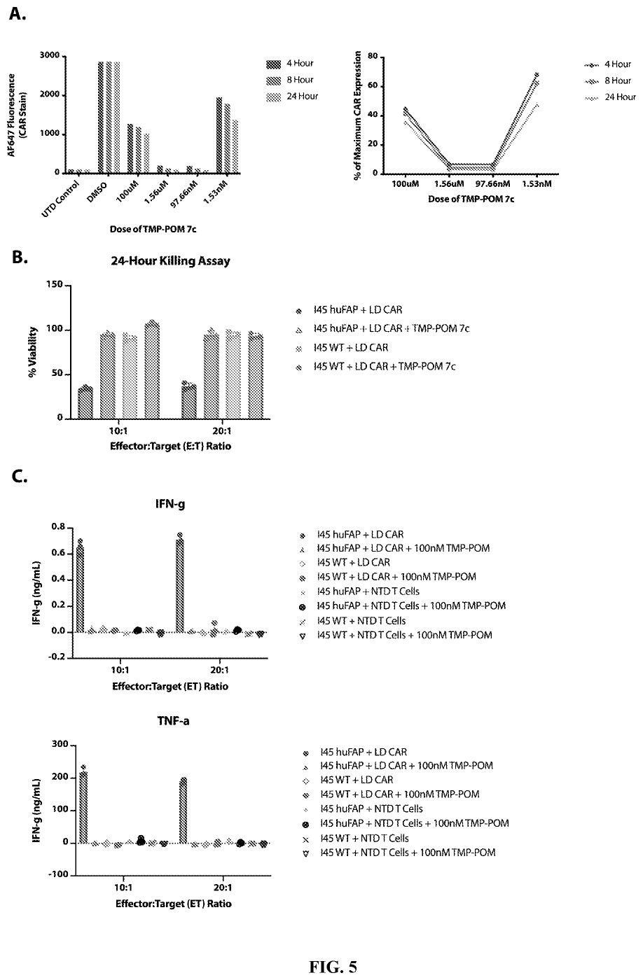

FIG. 5 degradation of CAR molecules from the surface of CAR-T cells using compound 7c.

FIG. 5A shows the kinetics and dose response of CAR downregulation following incubation of primary human FAP-eDHFR CAR T cells with compound 7c or DMSO is shown, evaluated at serial time points by flow cytometry. Data plotted as raw CAR stain fluorescence is shown in the left panel and percent of maximum CAR expression is shown in the right panel.

FIG. 5B shows compound 7c mediated CAR downregulation inhibits in vitro cytotoxic functional activity of FAP CAR T cells. In vitro killing assay was performed with FAP-eDHFR CAR T cells pre-incubated for 24 hours with 100 nM of compound 7c and following pre-incubation, the FAP-eDHFR CAR T cells were co-incubated with either target-expressing FAP⁺ 145 mesothelioma cell line or wild type (WT) for 24-hours at E:T ratios of 10:1 and 20:1. Data shown are in triplicates, error bars=SD.

FIG. 5C shows compound 7c mediated downregulation of FAP CAR inhibits T cell TNF-α and IFN-g release in vitro. The concentration of TNF-α and IFN-g in the supernatants following killing assay was determined by ELISA. Data shown are in triplicates, error bars=SD.

As can be seen, compound 7c leads to degradation of CAR molecules from the surface of CAR T cells, resulting in inhibition of their cytotoxic function.

### Example 10—“Cell Signaling Changes” Related to Degradation of CAR Molecules From the Surface of Jurkat Cells

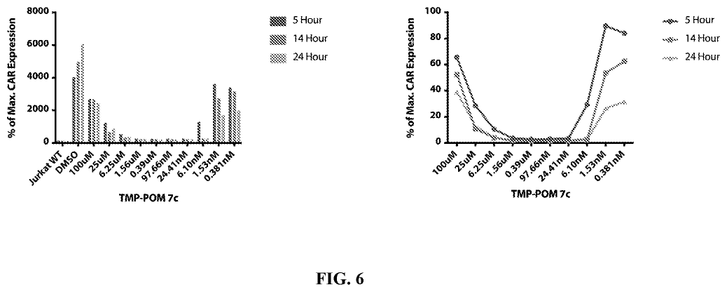

FIG. 6 shows “cell signaling changes” related to degradation of CAR molecules from the surface of Jurkat cells using compound 7c. FIG. 6 shows the kinetics and dose response of CAR downregulation with compound 7c or DMSO in Jurkat cells expressing FAP CAR-eDHFR fusion protein, evaluated at serial time points by flow cytometry. Data plotted as raw CAR stain fluorescence is shown in the left panel and percent of maximum CAR expression is shown in the right panel.

As can be seen, compound 7c leads to degradation of CAR molecules from the surface of Jurkat cells.

### Example 11—Imaging Cells Engineered with eDHFR with ¹⁸F-TMP

The K_{d} of ¹⁸F-TMP in human cells (HCT116) engineered with eDHFR was determined and found the Kd to be similar to parent TMP, −1 nM. ¹⁸F-TMP is shown below:

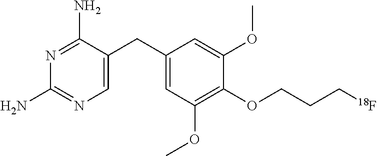 [SMILES: COc1cc(Cc2cnc(N)nc2N)cc(OC)c1OCCC[18F]]

Additionally, it has been demonstrated that in animal xenograft models expressing eDHFR, radiotracer derivatives of TMP (both [¹¹C] and [¹⁸F]) show promising time activity curves demonstrating the growth and maintenance of signal over time in the eDHFR expressing tumor rather than washout kinetics.

It has also been demonstrated that a handful of CAR T cells (11,000 cells/mm³) targeting the disialoganglioside GD2 invading into tumors can be imaged, and that correlative IHC/autoradiography captured the co-localization of the CAR T cells and the radio-signal.

### Example 12—Dose Response and Time Course of 7c in Jurkat^{eDHFR-YFP+} Cells

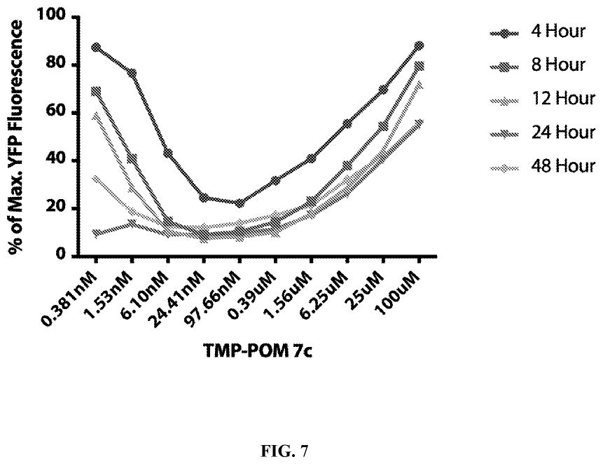

FIG. 7 shows dose response and time course of 7c in Jurkat^{eDHFR-YFP+} cell. Kinetics of YFP degradation by the lead compound 7c was characterized by incubating Jurkat^{eDHFR-YFP+} cells with serially diluted doses of 7c for 4, 8, 12, 24, and 48 h. The result demonstrated both dose and time-dependent YFP degradation by 7c, with robust degradation of YFP to 20% of maximum (no drug control) as early as 4 h.

### Example 13—Reversal Kinetics of YFP Degradation in Jurkat^{eDHFR-YFP+} Cells

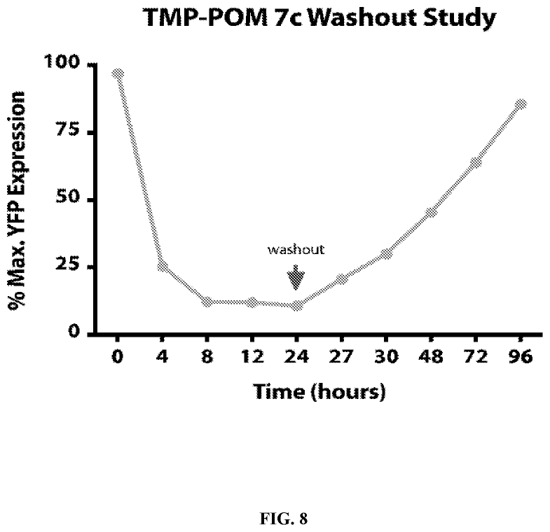

FIG. 8 shows reversal kinetics of YFP degradation in Jurkat^{eDHFR-YFP+} cells Jurkat^{eDHFR-YFP+} cells were incubated with 100 nM of compound 7c, and YFP expression was monitored at several time points by flow cytometry before and after drug washout. These data demonstrate that full return of YFP expression to baseline (no drug) following drug washout takes approximately 72 h.

### Example 14—Degradation of eDHFR-YFP in HEK293T (HEK293T^{eDHFR-YFP+}) Cells Analyzed by Western Blot with Anti-YFP Antibody

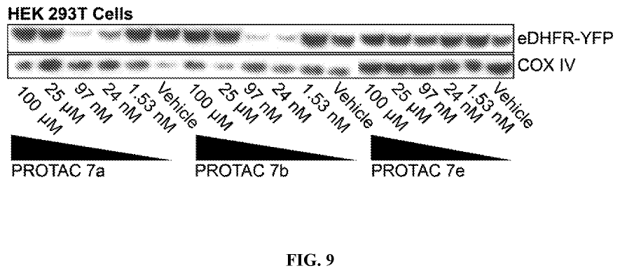

FIG. 9 shows degradation of eDHFR-YFP in HEK293T (HEK293T^{eDHFR-YFP+}) cells analyzed by Western blot with anti-YFP antibody. Dose response screen of compounds 7a, 7b, and 7e at 24 h. Compound 7a and 7b show optimal degradation of eDHFR-YFP between 97-24 nM, where 7e shows no degradation in 24 h.

### Example 15—Dose Response in HEK293T^{eDHFR-YFP+} Cells with Compound 7c at 24 h

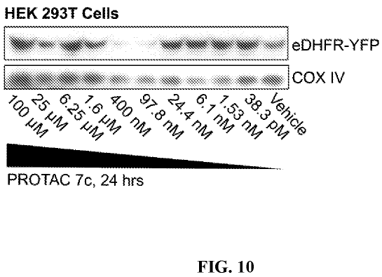

FIG. 10 shows dose response in HEK293T^{eDHFR-YFP+} cells with compound 7c at 24 h.

### Example 16—Time Course in HEK293T^{eDHFR-YFP+} Cells with Compound 7c at 6, 12 and 24 h

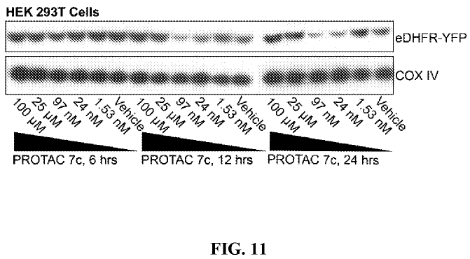

FIG. 11 shows time course in HEK293T^{eDHFR-YFP+} cells with compound 7c at 6, 12 and 24 h. eDHFR-YFP degradation observed between 97-24 nm at 12 h and decreases further in 24 h.

### Example 17—Western Blot Analysis of eDHFR-YFP Recovery in HEK293T^{eDHFR-YFP+} Cells Incubated with 100 nM 7c, Washed Twice with PBS, then Replenished with New Media

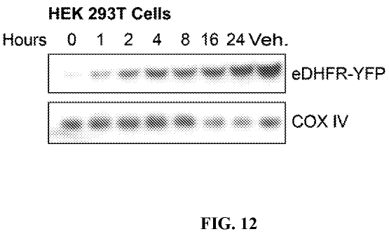

FIG. 12 shows Western blot analysis of eDHFR-YFP recovery in HEK293T^{eDHFR-YFP+} cells incubated with 100 nM 7c, washed twice with PBS, then replenished with new media. Protein degradation is reversed in as early as 4 h after drug removal from cell media.

### Example 18—Western Blot Characterization of Proteasome Degradation Mechanism in HEK293T^{eDHFR-YFP+} Cells

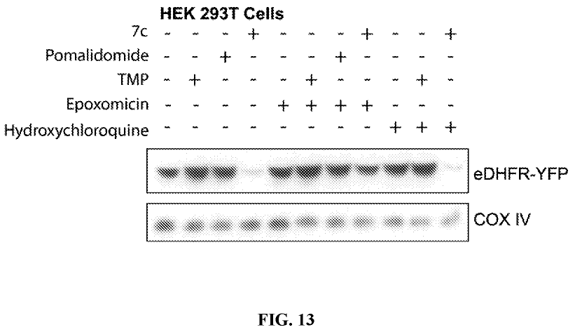

FIG. 13 shows western blot characterization of proteasome degradation mechanism in HEK293T^{eDHFR-YFP+} cells. HEK293T^{eDHFR-YFP+} cells were incubated with 500 nM Epoxomicin or 25 μM Hydroxychloriquine HCl for 1 h, followed by the addition of 100 nM of 7c, 25 μM TMP or 2.5 μM Pomalidomide, where cells were incubated for an additional 12 h. Epoxomicin blocks degradation induced by 7c, where Hydroxychloroquine HCl does not.

### Example 19—HEK293T^{eDHFR-YFP+} Cells were Incubated with Either 500 nM MLN4924 or 25 μM 3-Methyladenine for 1 h, Followed by the Addition of, 100 nM of 7c, 25 μM TMP or 2.5 μM Pomalidomide, where Cells were Incubated for an Additional 12 h

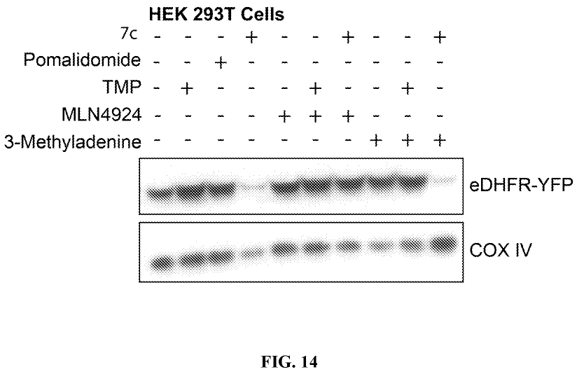

FIG. 14 shows HEK293T^{eDHFR-YFP+} cells were incubated with either 500 nM MLN4924 or 25 μM 3-Methyladenine for 1 h, followed by the addition of, 100 nM of 7c, M TMP or 2.5 μM Pomalidomide, where cells were incubated for an additional 12 h. MLN4924 blocks degradation induced by 7c, where 3-Methyladenine does not.

### Example 20—Characterization of 7f by Western Blot Analysis with Anti-YFP Antibody

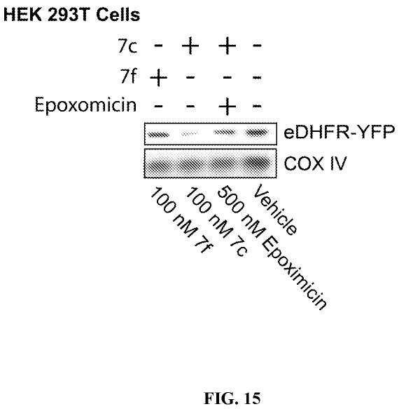

FIG. 15 shows characterization of 7f by Western blot analysis with anti-YFP antibody. HEK293T^{eDHFR-YFP+} Cells were incubated with 100 nM 7f, 100 nM 7c or 500 nM Epoxomicin for 24 h. Compound 7f does not induce eDHFR-YFP degradation, similar to Epoxomicin-blocked cells, where 7c alone causes eDHFR-YFP to degrade.

### Example 21—Dose Response in HEK293T^{+eDHFR-Lck} Cells with Compound 7c at 24 h

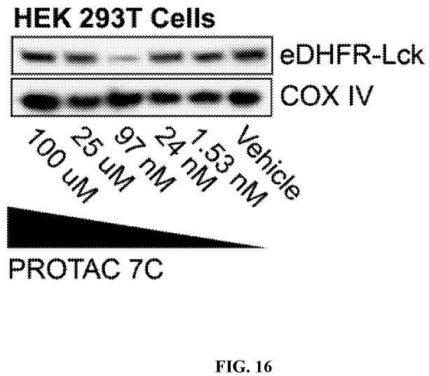

FIG. 16 shows dose response in HEK293T^{+eDHFR-Lck} cells with compound 7c at 24 h. Lck is a signaling molecule implicated in the formation of the major histocompatabiltiy complex (MHC) in immune cells. Western blot shows optimal degradation of eDHFR-Lck fusion protein at 97 nM.

### Example 22—Dose Response in HEK293T^{+eDHFR-RUX1} Cells with Compound 7c at 24 h

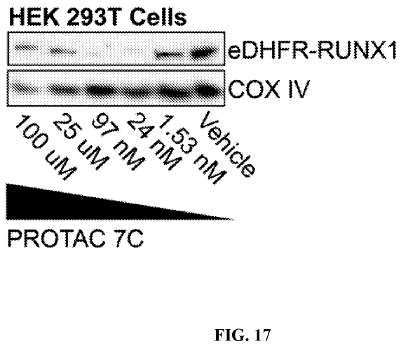

FIG. 17 shows dose response in HEK293T^{+eDHFR-RUX1} cells with compound 7c at 24 h. RUNX1 is a transcription factor that regulates the differentiation of hematopoietic stem cells. Western blot shows optimal degradation of eDHFR-RUNX1 fusion protein between 97-24 nM.

### Example 23—Dose Response in HEK293T^{+CD122-eDHFR} Cells with Compound 7c at 24 h

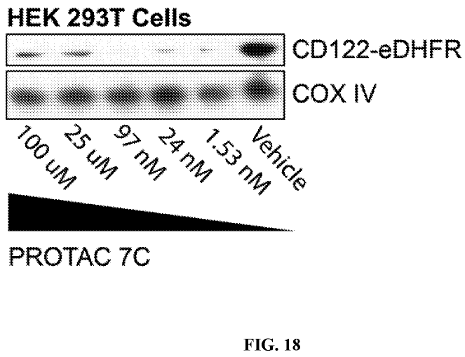

FIG. 18 shows dose response in HEK293T^{+CD122-eDHFR} cells with compound 7c at 24 h. CD122 is a transmembrane protein that is part of the IL2 receptor complex implicated in IL2-mediated T-cell signalling and is also implicated in the initiation of the MAPK, PI3K and JAK-STAT pathways. Western blot shows optimal degradation of CD122-eDHFR fusion protein between 97-1.53 nM.

### Example 24—OVCAR8 Cells Expressing eDHFR-Luc (OVCAR8eDHFR-Luc+) were Incubated with Compound 7c for 4-48 h

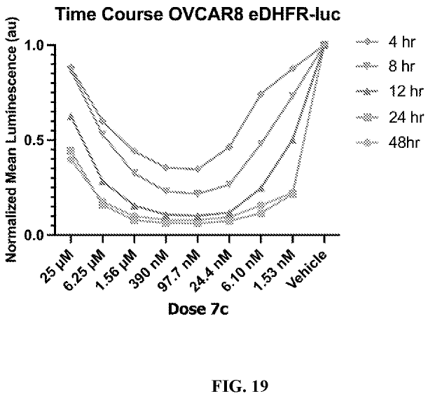

FIG. 19 shows OVCAR8 cells expressing eDHFR-luc (OVCAR8eDHFR-luc+) were incubated with compound 7c for 4-48 h. After the addition of Luciferin/complete media solution, luminescence was measured at the same time (t=0) by plate reader. 90% degradation of eDHFR-luc measured in 12 h between 390 and 24 nM, and 95% degradation of eDHFR-luc measured in 24 h between 390 and 24 nM.

### Example 25—TMP-POM 7c PROTAC Effectively Downregulates CAR in a Dose-Dependent and Reversible Manner

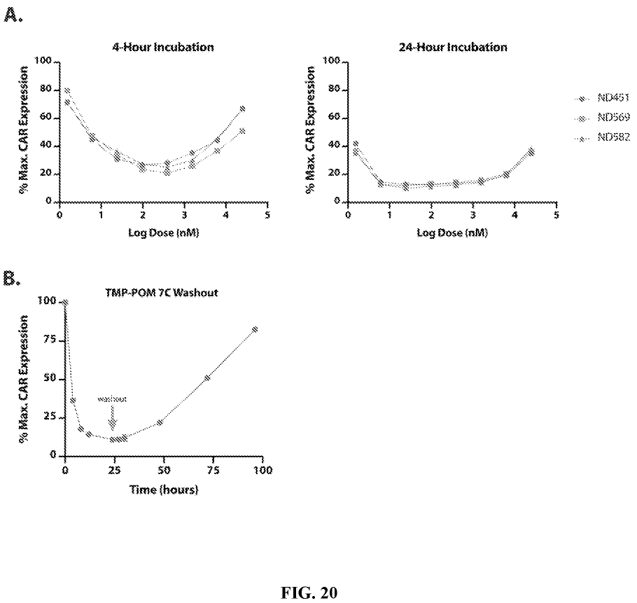

FIG. 20 shows TMP-POM 7c PROTAC effectively downregulates CAR in a dose-dependent and reversible manner.

FIG. 20A shows CAR expression across different concentrations of TMP-POM 7c at 4 and 24 hours post-incubation. 2×10⁵ primary human FAP-eDHFR DF CAR T cells were incubated with different concentrations of TMP-POM 7c for 4 and 24 hours in a 96-well plate, and their surface expression was evaluated by flow cytometry. The CAR T cells exhibited a dose-dependent downregulation of CAR expression from the surface of FAP CAR T cells, with effective downregulation to down to 20% of maximum CAR expression seen as early as 4 hours post-incubation with the PROTAC. Values are reported as percent of maximum CAR expression (i.e. relative to CAR T cells that were treated with vehicle control). Each color represents a different donor (n=3).

FIG. 20B shows kinetics of CAR downregulation with TMP-POM 7c and its reversibility. 6×10⁵ primary human FAP-eDHFR DF CAR T cells were incubated with 100 nM of TMP-POM 7c or vehicle control (DMSO) in a 12-well plate, and cells were sampled at different time points as indicated to assess for CAR expression by flow cytometry. Following 24-hour incubation with TMP-POM 7c, the cells were washed multiple times to remove the drug (red arrow), seeded on a new plate, and then again sampled serially following the drug washout. All cells were stained for CAR and fixed in 4% following sampling. The washout experiment demonstrated that it takes around 24 hours following washout of TMP-POM 7c for the CAR expression to start recovering and nearly 72 hours for the expression to return to baseline. n=3, data points are mean±SD.

### Example 26—Downregulation of CAR with TMP-POM PROTAC Inhibits CAR T Cell Signaling and its Cytotoxic Function Against Target Cells In Vitro

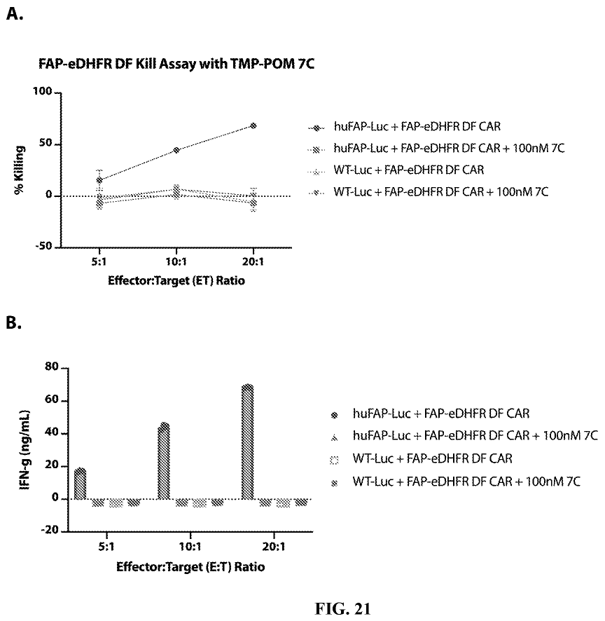

FIG. 21 shows downregulation of CAR with TMP-POM PROTAC inhibits CAR T cell signaling and its cytotoxic function against target cells in vitro.

FIG. 21A shows primary human FAP-eDHFR DF CAR T cells were pre-incubated with 100 nM of TMP-POM 7c or vehicle control (DMSO) for 24 hours and added to 145 mesothelioma cells expressing human FAP and optical protein luciferase (I45 huFAP-Luc) at varying effector-to-target (E:T) ratios. Following overnight co-incubation of target cells and effector FAP-eDHFR CAR T cells, luminescence was read on a plate reader to assess for target cell viability. The killing assay demonstrated complete abrogation of FAP-eDHFR DF CAR T cell-mediated specific cytotoxicity against FAP-expressing target cells in presence of TMP-POM 7c.

FIG. 21B shows the cell supernatant from the above killing assay was collected and the level of IFNγ secretion by FAP CAR-eDHFR CAR T cells was determined by ELISA. The measured IFNγ secretion mirror the pattern seen in the killing assay and confirmed inhibition of CAR T cell signaling in presence of TMP-POM 7c. n=3, data points are mean±SD.

### Example 27—TMP-POM 7c can Modulate the Cytotoxic Activity of FAP-eDHFR DF CAR T Cells in a Dose-Dependent Manner with TMP-POM 7c

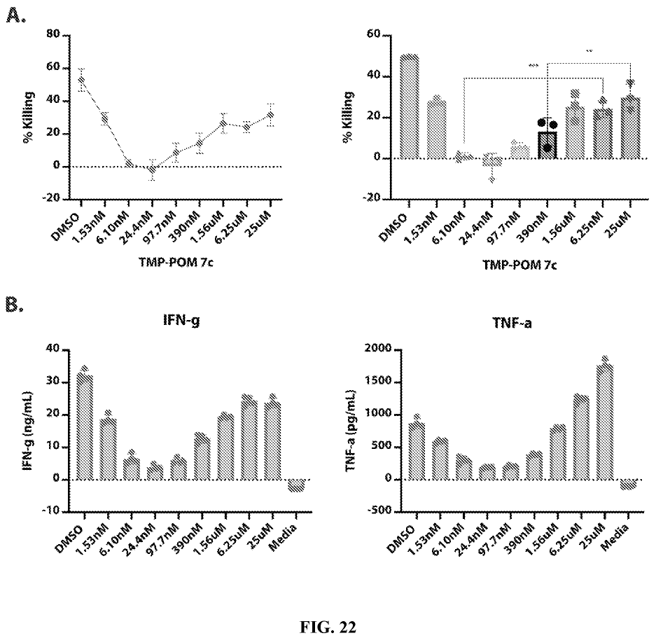

FIG. 22 shows TMP-POM 7c can modulate the cytotoxic activity of FAP-eDHFR DF CAR T cells in a dose-dependent manner with TMP-POM 7c.

FIG. 22A shows primary human FAP-eDHFR DF CAR T cells were pre-incubated with TMP-POM 7c overnight at different concentrations, and were added to the target I45huFAP-Luc cells at a 10:1 effector-to-target (E:T) ratio. TMP-POM 7c was added to each well to maintain the drug at the indicated concentration. Following overnight co-incubation of target cells and effector FAP-eDHFR CAR T cells, luminescence was read on a plate reader to assess for target cell viability. The assay showed that the FAP-eDHFR DF CAR T cell activity against FAP-expressing target cells was tunable in a dose-dependent manner. n=3, data points are mean±SD, and groups were compared using a one-way ANOVA with Tukey's multiple comparison test. **p=0.0037, ***p=0.0001.

FIG. 22B shows the cell supernatant from the above killing assay was collected and the level of IFNγ and TNFα secretion by FAP CAR-eDHFR CAR T cells was determined by ELISA. The measured IFNγ and TNFα secretion supported the killing assay p the pattern seen in the killing assay and confirmed inhibition of CAR T cell signaling in presence of TMP-POM 7c. n=3, data points are mean±SD.

### Example 28—In Vitro Characterization of FAP-eDHFR DF CAR Constructs

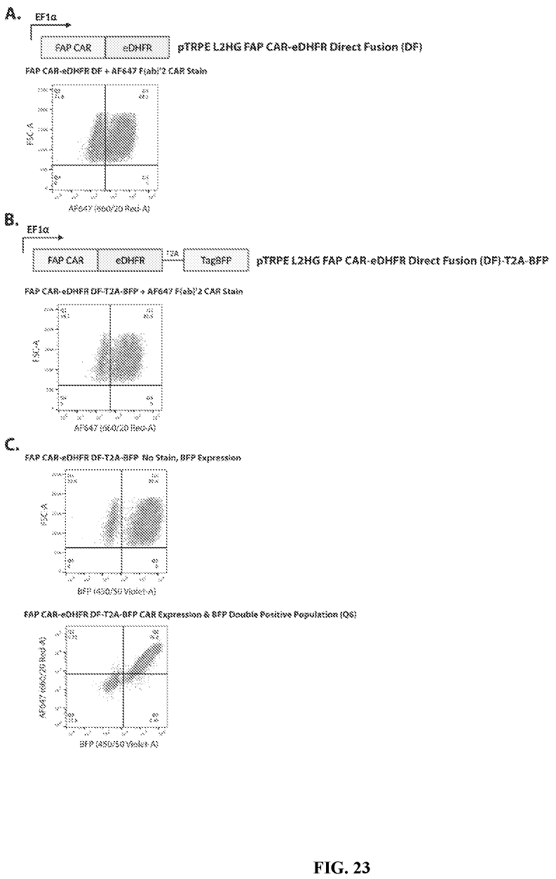

FIG. 23 shows In vitro characterization of FAP-eDHFR DF CAR constructs.

FIG. 23A shows primary human T cells were transduced with pTRPE L2HG FAP CAR-eDHFR direct fusion (DF) construct, and the transduction efficiency was assessed by staining for CAR expression using Alexa Fluor® 647 AffiniPure F(ab′2) fragment goat anti-mouse IgG. CAR+ population (˜81.5%) were sorted on the AF647 stain for downstream assays.

FIG. 23B shows primary human T cells were transduced with pTRPE L2HG FAP CAR-eDHFR DF-T2A-BFP construct, and the transduction efficiency was assessed and sorted as described above.

FIG. 23C shows primary human T cells transduced with the pTRPE L2HG FAP-eDHFR DF-T2A-BFP construct were assessed using 45/50 Violet-A filters on a flow cytometer to confirm its BFP expression.

### Example 29—Dose Response Assay with N-Methyl 7c (7f)

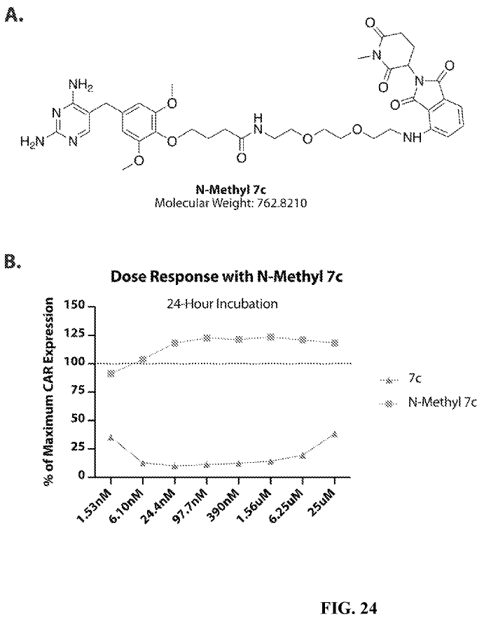

FIG. 24 shows dose response assay with N-Methyl 7c (7f).

FIG. 24A shows structure of N-Methyl 7c (7f).

FIG. 24B shows N-Methyl 7c (7f) dose response assay in primary human FAP-eDHFR DF CAR T cells. 2×10⁵ primary human FAP-eDHFR DF CAR T cells were incubated with different doses of TMP-POM 7c or N-Methyl 7c (7f) for 24 hours in a 96-well plate, and their surface expression was evaluated by flow cytometry. The result demonstrated that the incubation of CAR T cells with N-Methyl 7c (7f) does not result in downregulation of CAR expression from their surface, highlighting that the CAR regulation observed in 7c is specifically mediated by the pomalidomide (POM) domain of the compound.

### Example 30—Comparison of Cytotoxic Function of Different eDHFR-Expressing FAP CAR T Cells

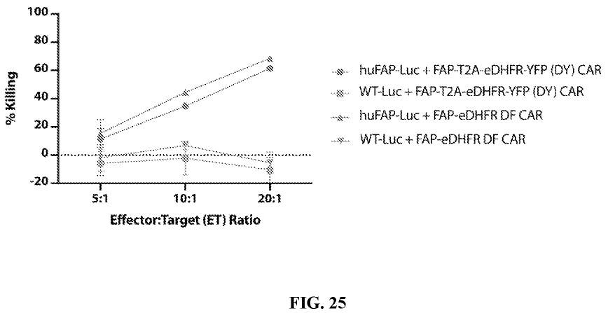

FIG. 25 shows comparison of cytotoxic function of different eDHFR-expressing FAP CAR T cells. Target-specific cytolytic activity of human FAP-eDHFR DF CAR T cells expressing a direct fusion of CAR and eDHFR was compared to FAP-T2A-eDHFR-YFP (DY) CAR T cells that have a cytosolic expression of eDHFR (i.e. the CAR domain and eDHFR are separated by a T2A site and therefore are not directly fused). The two types of effector CAR T cells were co-incubated with 145 WT-Luc or 145 huFAP-Luc target cells overnight at varying E:T ratios, and luminescence was read on a plate reader the next day to assess for target cell viability. The result demonstrated that the direct fusion of eDHFR to the C-terminus of the CAR domain does not inhibit signaling of CAR T cells and subsequent cytolytic activity against target cells, and the degree of cytotoxicity elicited by the DF CAR T cells was comparable to the DY CAR T cells that have a cytosolic expression of eDHFR. n=3, data points are mean±SD.

### Example 31—Downregulation of CAR by TMP-POM 7C PROTAC is a Proteosome-Mediated Degradation Process

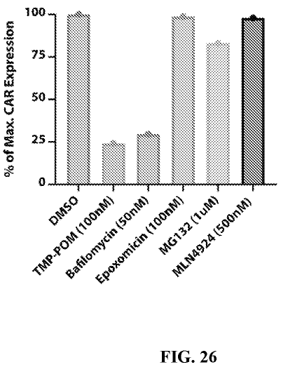

FIG. 26 shows downregulation of CAR by TMP-POM 7C PROTAC is a proteosome-mediated degradation process. 5×10⁵ primary human FAP-eDHFR DF CAR T cells were pre-incubated with 50 nM bafilomycin (lysosome inhibitor), 100 nM epoxomicin (proteosome inhibitor), 1 uM MG132 (proteosome inhibitor), or 500 nM MLN4924 (neddylation inhibitor) for 1 hour. Following pre-incubation, the cells were incubated with 100 nM 7c for 4 hours and their CAR expression was assessed on a flow cytometer. The inhibitor test showed that inhibition of proteasome and the neddylation pathway blocked TMP-POM 7c-mediated degradation of CAR, whereas inhibition of the endosome-lysosome pathway had no impact on TMP-POM 7c function.

### Example 32—Evaluation of the “Imageability” of FAP-eDHFR Direct Fusion (DF) CAR T Cells

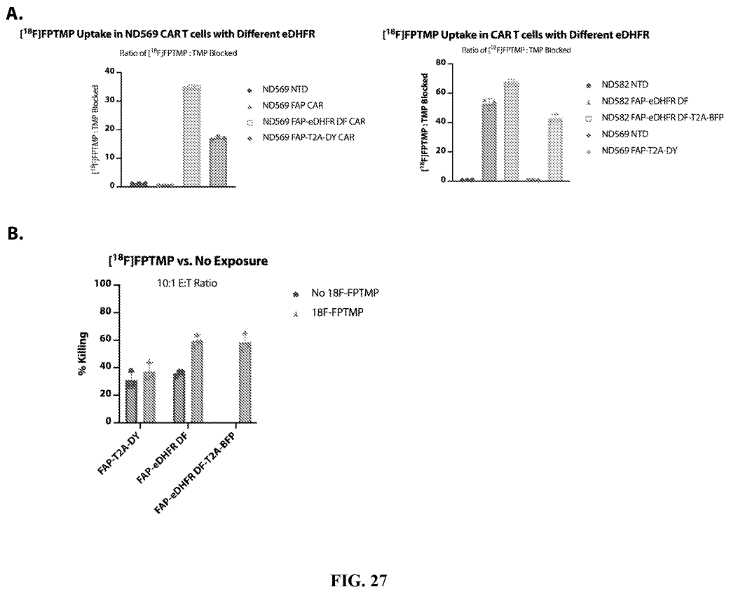

FIG. 27 shows evaluation of the “imageability” of FAP-eDHFR Direct Fusion (DF) CAR T cells.

FIG. 27A shows 1×10⁶ primary human FAP-eDHFR DF CAR T cells and FAP-T2A-eDHFR-YFP (DY) CAR T cells were incubated with [18F]FPTMP (2×10⁶ cpm per 1×10⁶ cells) for 1 hour at 37° C. in the presence or absence of excess, unlabeled TMP (5 μM). The experiment demonstrated preserved in vitro uptake of radiolabeled TMP in the FAP-eDHFR DF CAR T cells to the level that is superior to what is observed in the FAP-T2A-eDHFRYFP (DY) CAR T cells (original imageable CAR construct, Sellmyer et al. 2020), suggesting that the direct fusion of eDHFR to the CAR domain does not affect its binding nor its imageability with radiolabeled TMP. n=3, data points are mean±SD.

FIG. 27B shows cytotoxic function of FAP-eDHFR DF CAR T cells that were exposed to [¹⁸F]FPTMP (in the above uptake study) were compared to FAP-eDHFR DF CAR T cells that were not treated with radiolabeled TMP by co-incubating the cells with the target 145 huFAP cells at a 10:1 E:T ratio overnight. The killing assay demonstrated that exposure of CAR T cells to [¹⁸F]FPTMP-induced radiation does not affect the effectors' ability to signal and kill target cells. Please note that there was no sample of FAP-eDHFR DF-T2A-BFP CAR T cells that were exposed to [¹⁸F]FPTMP for this experiment. n=3, data points are mean±SD.

1. A compound of formula (I): 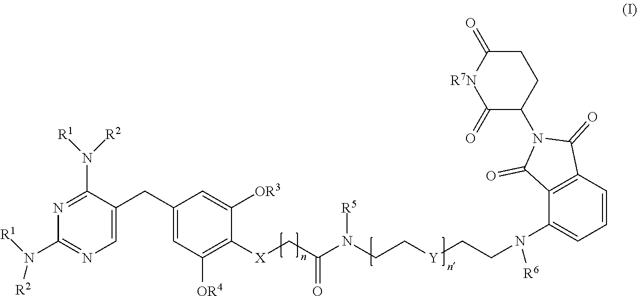
or a pharmaceutically acceptable salt thereof, wherein:
X is —O—, —S—, —CR¹R²— or —NR¹—;
Y is —O—, —S—, —CR¹R²— or —NR¹—;
each of R¹, R², R³, R⁴, R⁵, R⁶ and R⁷ is independently selected from hydrogen or C1-C6 alkyl;
n is 1, 2, 3, 4, 5, or 6; and
n′ is 1, 2, 3, 4, 5 or 6.

2. (canceled)

3. (canceled)

4. (canceled)

5. The compound of claim 1, wherein at least one of R¹, R², R³, R⁴, R⁵, R⁶ and R⁷ is hydrogen.

6. The compound of claim 1, wherein each of R¹, R², R³, R⁴, R⁵, R⁶ and R⁷ is hydrogen.

7. The compound of claim 1, wherein at least one of R¹, R², R³, R⁴, R⁵, R⁶ and R⁷ is C₁-C₆ alkyl.

8. The compound of claim 1, wherein each of R¹, R², R³, R⁴, R⁵, R⁶ and R⁷ is C₁-C₆ alkyl.

9-24. (canceled)

25. The compound of claim 1, wherein the compound of formula (I) is: 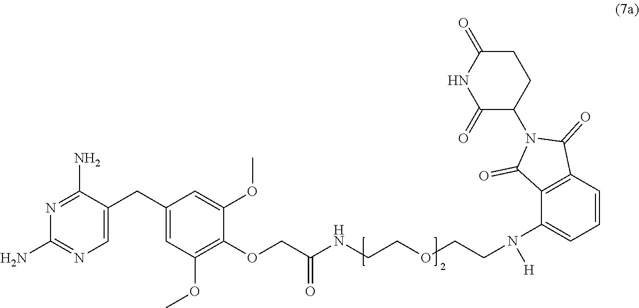
or a pharmaceutically acceptable salt thereof.

26. The compound of claim 1, wherein the compound of formula (I) is: 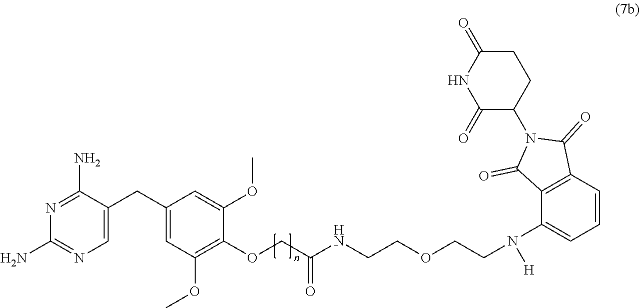
or a pharmaceutically acceptable salt thereof.

27. The compound of claim 1, wherein the compound of formula (I) is: 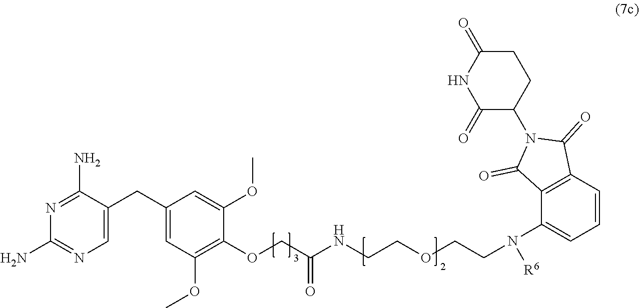
or a pharmaceutically acceptable salt thereof.

28. The compound of claim 1, wherein the compound of formula (I) is: 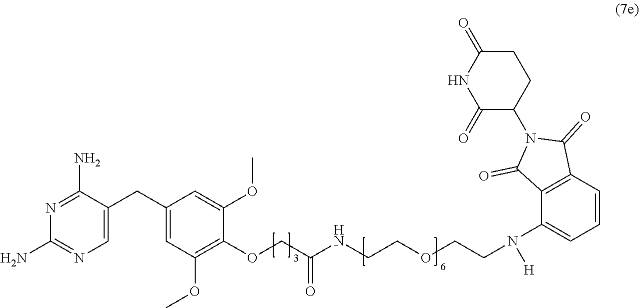
or a pharmaceutically acceptable salt thereof.

29. The compound of claim 1, wherein the compound of formula (I) is: 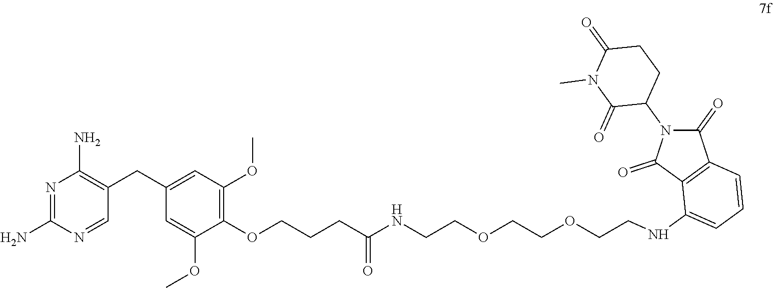 [SMILES: COc1cc(Cc2cnc(N)nc2N)cc(OC)c1OCCCC(=O)NCCOCCOCCNc1cccc2c1C(=O)N(C1CCC(=O)N(C)C1=O)C2=O]
or a pharmaceutically acceptable salt thereof.

30. A method of regulating protein expression in a mammalian cell, comprising: contacting dihydrofolate reductase enzyme (DHFR) with a compound of formula (I) of claim 1, or a pharmaceutically acceptable salt thereof.

31. (canceled)

32. The method of claim 30, wherein the DHFR is Escherichia coli dihydrofolate reductase enzyme (eDHFR).

33. The method of claim 30, further comprising in vivo imaging of the mammalian cell comprising the steps of:
(a) administering 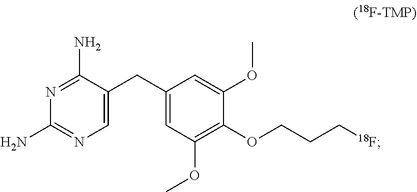
and
(b) imaging in vivo distribution of the ¹⁸F-TMP by positron emission tomography (PET) scanning.

34. A method of degrading a protein of interest, comprising: contacting the protein of interest with a compound of formula (I) of claim 1, or a pharmaceutically acceptable salt thereof.

35. The method of claim 34, wherein the protein of interest is a kinase, a cytokine, an immunotherapy protein, a chimeric protein, a structural protein, a transcription factor, a hormone, a growth factor, an immunoglobulin, an antibody, an immunoglobulin-like domain-containing molecule, an ankyrin, a fibronectin domain-containing molecule, or an Fc-fusion protein.

36-40. (canceled)

41. A degraded protein made by the method of claim 34.

42. A method of making an engineered cell, comprising: fusing a protein of interest to a dihydrofolate reductase enzyme (DHFR) with a compound of formula (I) of claim 1, or a pharmaceutically acceptable salt thereof.

43. (canceled)

44. (canceled)

45. (canceled)

46. An engineered cell made by the method of claim 42.

47-51. (canceled)

52. A kit, comprising: a dihydrofolate reductase enzyme (DHFR) construct and a compound of formula (I) of claim 1, or a pharmaceutically acceptable salt thereof.

53-58. (canceled)

59. A method of in vivo imaging of a mammalian cell comprising the steps of:
(a) administering 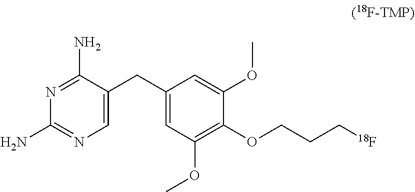 [SMILES: COc1cc(Cc2cnc(N)nc2N)cc(OC)c1OCCC[18F]]
to a subject; and
(b) imaging in vivo distribution of the ¹⁸F-TMP by positron emission tomography (PET) scanning.

60-65. (canceled)
